(19) United States
(12) Patent Application Publication
(10) Pub. No.: US 2025/0302694 A1
(43) Pub. Date: Oct. 2, 2025

(54) System and Method of Secure Pill Dispensing

(71) Applicant: Secretbox Inc., Long Beach, CA (US)

(72) Inventor: Sofia Esteves Carvalho, San Pedro, CA (US)

(21) Appl. No.: 19/091,767

(22) Filed: Mar. 26, 2025

Related U.S. Application Data
(60) Provisional application No. 63/569,933, filed on Mar. 26, 2024.

(51) Int. Cl. A61J 1/03; A61J 7/04; G16H 20/13; G16H 30/40; G16H 40/67

(52) U.S. Cl. CPC A61J 1/03; A61J 7/0418; A61J 7/0427; G16H 20/13; G16H 30/40; G16H 40/67

(57) ABSTRACT

In one aspect, a system for secure pill dispensing is presented. The system includes a pill container, such as a pill bottle. The pill bottle includes a dispensing compartment including a dispenser. The dispenser is operable to dispense a pill from the pill bottle. The pill bottle includes a communication module configured to communicate with software that is executable by a computing device. The communication module receives input from the software. The pill bottle includes a controller in communication with the dispensing compartment and the communication module. The controller is programmed to activate the dispenser based on the input received by the communication module.

### CROSS-REFENCE TO RELATED APPLICATIONS

This application claims the benefit of U.S. Provisional Application No. 63/569,933, filed Mar. 26, 2024, entitled “System and Method of Secure Pill Dispensing,” which is incorporated by reference.

### TECHNICAL FIELD

The following disclosure is directed to pill dispensing systems or pill containers or pill bottles. In particular, the present disclosure is directed to systems and methods for secure pill dispensing.

### SUMMARY OF THE DISCLOSURE

In one aspect, a system for secure pill dispensing is presented. The system includes a pill container or pill bottle. The pill container includes a dispensing compartment including a dispenser. The dispenser is operable to dispense a pill from the pill container. In embodiments, the pill container includes a communication module configured to communicate with software that is executable by a computing device. The communication module receives input from the software. In embodiments, the pill container includes a controller in communication with the dispensing compartment and the communication module. The controller is programmed to activate the dispenser based on the input received by the communication module.

In another aspect, there is a method of secure pill dispensing in a pill container. In embodiments, the pill container includes a communication module, a controller, and a dispenser for the pill container coupled to the controller. In embodiments, the method includes receiving, at the communication module of a pill container, user input from a user to dispense a pill of the pill container. The method includes determining by the controller or software linked to the pill container in a computing device whether or not to dispense the pill based on a) a prescription for the pill and b) a last dispensing of a prior pill communicated by the pill bottle. In embodiments, the method includes dispensing, by the dispenser of the pill container, the pill to the user only if the determination allows the pill to be dispensed.

In another aspect, a pill container is presented. The pill container includes a housing storing at least a pill. The housing stores a power supply. In embodiments, the housing stores a communication module configured to communicate with a computing device. The communication module receives a request to dispense the at least a pill from the computing device. In embodiments, the housing stores a dispensing compartment positioned at a bottom of the housing. The dispensing compartment includes a dispenser that dispenses the at least a pill. In embodiments, the pill container includes an electrical communication with the communication module and the dispenser. The controller or software linked to the pill container determines whether or not to dispense the pill based on a) a prescription for the pill and b) a last dispensing of a prior pill communicated by the pill container.

In other embodiments, a pill container is disclosed. The pill container may include a hollow structure comprising one or more side surfaces, a top structure opening, a bottom structure opening, and a side structure opening that extends through one of the one or more side surfaces. The pill container may further include a rotating surface configured to be positioned within the hollow structure and above the side structure opening and to rotate about a first axis that extends perpendicular to the one or more side surfaces and through the top opening and bottom opening, wherein the rotating surface includes one or more slots that extend through the rotating surface. The pill container may also include an enclosure configured to attach at the bottom structure opening of the hollow structure, wherein the enclosure is detachable from the hollow structure and houses an electronic system configured to control rotation of the rotating surface.

In embodiments, the one or mor slots are positioned along a ring around the first axis, the pill container further comprising a funnel positioned within the hollow structure above the rotating surface, wherein a top funnel opening of the funnel spans a width between the one or more surfaces and a bottom funnel opening of the funnel is aligned above the ring of one or more slots.

In aspects, the enclosure includes a sensor embedded in a top portion of the enclosure, wherein the sensor is aligned with the bottom funnel opening of the funnel when the enclosure is attached to the hollow structure, the sensor configured to capture signals indicative of a pill moving through the side structure opening of the hollow structure.

In embodiments, the sensor is a light sensor, pressure sensor, or a motion sensor.

In some aspects, a lid is configured to attach at the top structure opening via a locking mechanism, wherein the lid is rotatable around the first axis while attached at the top structure opening.

In further aspects, the one or more slots include at least two slots, each slot a different size from the other slots of the one or more slots.

In further embodiments, a first slot of the one or more slots is configured to allow pills of a first size and a second size to move through the first slot and a second slot of the one or more slots is smaller and is configured to allow pills of the first size to move through the second slot and to prevent pills of the second size from moving through the second slot.

In embodiments, the electronic system is configured to receive a signal from a client device and cause the rotating surface to rotate such that one or more pills are dispensed through the side structure opening in response to receiving the signal.

The disclosure also includes a method comprising various steps. The steps include: 1) sending, to a pill container from a client device, a signal to dispense a first pill; 2) causing a camera of the client device to capture image data; 3) inputting the image data to a machine-learned model, wherein the machine-learned model is trained to determine whether the image data depicts a user associated with the pill container swallowing the first pill, 4) receiving, from the machine-learned model, an indication of whether the image data depicts the user swallowing the first pill; and 5) in response to determining that the image data depicts the user swallowing the first pill, storing a first set of compliance data in association with an identifier of the user, wherein the first set of compliance data indicates that the user swallowed the first pill.

In aspects, the method also includes steps of: determining that a current time at the client device is in a time window for dispensing a second pill; and in response to determining that a most recently stored set of compliance data is indicative of the user swallowing a pill, sending a second signal to dispense a second pill.

In aspects, the method further includes: in response to determining that the image data does not depict the user swallowing the first pill, sending an alert to a second client device associated with a medical entity; and storing a second set of compliance data in association with the identifier of the user, wherein the second set of compliance data indicates that the user did not swallow the first pill.

In further embodiments, the method includes determining that a current time at the client device is in a time window for dispensing a second pill; and in response to determining that a most recently stored set of compliance data indicates that the user did not swallow a pill, sending an alert to a medical entity associated with the user.

In additional aspects, the indication of whether the image data depicts the user swallowing the pill comprises indication of a likelihood that the user swallowed the first pill, and the method further comprises triggering an alert in response to the likelihood being outside a threshold tolerance.

In further embodiments, a first class of pills are associated with a lower threshold tolerance than a second class of pills.

In other embodiments, the method includes training the machine-learned model on a first set of labeled image data, wherein image data depicting one or more of a set of throat movements is labeled as depicting swallowing, and image data not depicting one or more of the set of throat movements is labeled as not depicting swallowing; and training the machine-learned model on a second set of labeled image data, wherein image data of the user is labeled as depicting the user and image data that is not of the user is labeled as not depicting the user.

In additional aspects, the method includes receiving, via a user interface at the client device, an indication that the user lost the pill container; and sending, to a medical entity associated with the user, an indication that the user lost the pill container.

In aspects, the method includes receiving, via a user interface at the client device, an indication that the user lost the pill container; requesting, from the pill container, a current location of the pill container; comparing the current location of the pill container to a current location of the client device; and in response to the current location of the pill container being outside of a threshold distance from the current location of the client device, sending a request for new pills to a second client device associated with a medical entity.

The disclosure also includes a method comprising various steps. The steps include: 1) receiving, at a pill container from a client device, an indication to dispense a pill from the pill container, wherein the pill container contains at least one pill in a first enclosure, the first pill positioned above a rotating surface within the pill container; causing the rotating surface within the pill container to rotate, wherein the rotating surface includes a plurality of slots, wherein at least one slot is wide enough for the pill to move through the slot; and in response to detecting, via a sensor configured at an opening of the pill container, a signal indicative of a pill moving through the opening, wherein the opening is below the rotating surface: sending, to the client device, an indication that the pill was dispensed; and stopping rotation of the rotating surface.

In aspects, the method includes that each slot of the plurality of slots has a different width than the other slots.

In embodiments, the method includes sending, for integration in an electronic record of an electronic record system associated with a medical entity, a notification that the pill was dispensed at the pill container.

In embodiments, the method includes in response to determining that a user associated with the pill container is not in compliance with one or more medication management rules, locking the rotating surface, wherein locking the rotating surface prevents the rotating surface from rotating until a notification from a medical entity associated with a user of the client device is received.

The method may also comprise in response to detecting the signal indicative of a pill moving through the opening, increasing a count of pills dispensed at the pill container; and in response to determining that that count has exceeded a threshold, locking the rotating surface, wherein locking the rotating surface prevents the rotating surface from rotating until a notification from a medical entity associated with a user of the client device is received.

In some cases, the method comprises in response to determining that that count has exceeded a threshold, sending an alert to a second client device of a medical entity associated with a user of the client device is received, wherein the alert indicates the count.

The disclosure also includes a pill container system comprising a hollow structure comprising one or more side surfaces, a top structure opening, a bottom structure opening, a side structure opening that extends through one of the one or more side surfaces, a door covering the side opening. There may also be a locking lid attachable to the pill container to cover the top structure opening of the hollow structure. There may also be an enclosure configured to attach at the bottom structure opening of the hollow structure, wherein the enclosure is detachable from the hollow structure and houses an electronic system configured to control opening and closing of the door for dispensing pills from the side structure opening.

In embodiments, the electronic system comprises a processor; and a non-transitory computer-readable storage medium storing code that, when executed, causes a communication module to receive an indication that a computing device verified the identity of a user associated with the pill container system; and cause the door to open such that a pill is dispensed from the pill container system, wherein the communication module is configured to communicate with software that is executable by the computing device and receive input from the software.

In aspects, the indication is determined based on image or video data captured by an imaging sensor, wherein the imaging sensor is configured to capture image or video verifying the identity of the user.

In aspects, the imaging sensor is at the computing device.

In aspects, the code, when executed, further causes the communication module to input, to an artificial intelligence system, the image or video captured by the imaging sensor, wherein the artificial intelligence system is configured to verify whether input image or video depicts the user, the artificial intelligence system trained on image or video depicting the user; and receive, from the artificial intelligence system, the indication.

In embodiments, the system further includes a light sensor configured to capture light indicative of a pill being dispensed from the pill container system.

In embodiments, the system further comprises an identifier that allows registration of the pill container system with a prescription for pills of the pill container system.

In aspects, an indicator operable to indicate to the user that a pill is ready to be dispensed.

In embodiments, the indicator comprises one or more of a visual notification, vibrational notification, auditory notification, and haptic notification.

In embodiments, the code, when executed, further causes a compliance module to cause the imaging sensor to record a video of the user taking a pill dispensed from the pill container system; and determine a compliance to the prescription.

In some cases, the code, when executed, further causes the compliance module to analyze the video of the user taking the pill through a machine-learning model to assess compliance of the user with the prescription and provide an output related to the compliance.

In additional embodiments, the communication module receives an input from the software and conveys output based on the software to a controller of the electronic system.

In further embodiments, the input to the communication module comprises one of a number of pills to dispense, timing restrictions of dispensing, maximum daily number of pills to be dispensed, minimum time between dispensing of pills, emergency override for dispensing a number of pills.

In some cases, the software receives the prescription from a pharmacy provider data management (PDM) system, wherein the PDM system provides an identifier that allows for registration of the pill container system with the prescription for the pill.

In embodiments, there is a dispensing compartment that comprises a motor in electrical communication with the controller, and a conveyer in mechanical communication with the motor, wherein the controller moves the conveyer through activation of the motor.

In some cases, at least one of the controller and the software is configured to provide details of pill dispensing and/or compliance upon request by the user, a prescriber, or a third party.

In aspects, the pill container system further comprises a manual override button that when activated causes the controller to activate a dispenser during an unauthorized dispensing time.

In embodiments, the input from the software comprises an override command, wherein the communication module conveys the override command to the controller, wherein the controller activates the dispenser based on the override command during an unauthorized dispensing time.

In aspects, the pill container system further comprises one or more of an onboard memory that records data pertaining to the dispensing of pills and the software being in communication with a storage device that records the data pertaining to the dispensing of the pills.

In aspects, the data comprises one or more of events of an emergency override, dispensing of a pill, time between dispenser activation, time of dispenser activation, number of times of dispenser activation, or quantity of pills dispensed.

In aspects, one or more of the controller and the software are configured to alert an entity of non-compliance with the prescription.

In some embodiments, the entity is the user, the prescriber, a family member of the user, a medical professional, or a pharmacy.

In embodiments, the software is controlled or viewed by the third party.

In aspects, one or more of the software and controller are configured to send reminders to the user to take the pill.

In embodiments, the reminder is sent from the third party to the computing device via the software or is automatically generated and sent to the computing device via the software.

In aspects, the software is programmed to keep track of multiple prescriptions.

In aspects, the software is programmed to link multiple identities of multiple pill container systems to multiple prescriptions.

In embodiments, the pill container system further comprises a display configured to display data received generated by one or more of the controller and the software.

In embodiments, the data the display shows is one or more of a pill count, color coded reminder indicating when a next dose can be taken, or countdown to next dose.

In some cases, the data shown in the display pertains to a detection or non-detection of an instruction event.

In additional embodiments, data generated by one or more of the controller and the software are used in compliance reports.

In embodiments, the code that when executed further causes the compliance module to receive an input to dispense the pill; determine whether or not to dispense the pill based on a) the prescription for the pill and b) a last dispensing of a prior pill communicated by the pill container system; and dispense, by the dispenser, the pill only if the determination allows the pill to be dispensed.

In aspects, the last dispensing of a prior pill communicated by the pill container system is earlier to a last dispensing of a prior pill, earlier to one or more pill dispensings, or earlier to all prior pill dispensings.

In aspects, dispensing comprises activating a motor of the dispenser through the controller; and moving a conveyor of the dispenser with the pill thereon using the motor to dispense the pill.

In embodiments, wherein the code, when executed, further causes the compliance module to record, by an on-board memory of the pill container system, software in communication with the communication module, or both, data pertaining to dispensing of the pill

In additional aspects, the code that when executed further causes a compliance module to notify the user, through the indicator of the pill container system, that the pill is ready to be dispensed.

In aspects, the code, when executed, further causes the compliance module to locate, by a location device of the pill container system, a location of the pill container system; and authorize, by the controller of the pill container system, dispensing of the pill based on the location of the pill container system.

In aspects, the code, when executed, further causes the compliance module to determine, by a weight sensor of the pill container system, a quantity of pills stored in the pill container system.

In aspects, the code, when executed, further causes the compliance module to manually override, by the manual override button of the pill container system, a dispensing of the pill during the unauthorized dispensing time.

In aspects, the code, when executed, further causes the compliance module to override, from the input from the software, dispensing of the pill during the unauthorized dispensing time using the controller.

In aspects, the code, when executed, further causes the compliance module to alert, by one or more of the controller the software, the entity of user non-compliance with a prescription.

In aspects, the code, when executed, further causes the compliance module to send reminders to the user to take a pill through one or more of the software and the controller.

In aspects, the code, when executed, further causes the compliance module to keep track of multiple prescriptions via the software.

In embodiments, the system includes a sensor in electrical communication with the controller, wherein the sensor detects an intrusion event and communicates sensor data of the intrusion event to the controller.

In embodiments, the system includes comprising a kinetic power generator, wherein the kinetic power generator converts kinetic energy into electrical energy and transfers the electrical energy to the power supply.

In embodiments, the system includes a speaker in communication with the controller, wherein the speaker is configured to output a sound.

In embodiments, the controller is configured to activate the speaker in response to a request from the software to locate the pill container system.

In embodiments, the system includes an LED indicator that is activated by the controller in response to a request from the software to locate the pill container system.

In aspects, the software is programmed to communicate with the communication module to locate a position of the pill container system when the pill container system is lost.

In aspects, the software is programmed to display a visual indication of a location of the pill container system in reference to the location of the computing device based on the communication with the communication module.

In aspects, the visual indication is an arrow, radar, or augmented reality (AR) display of the pill container system.

### BRIEF DESCRIPTION OF THE DRAWINGS

FIG. 1 illustrates a block diagram of an exemplary embodiment of a system for secure pill dispensing, according to one or more embodiments;

FIG. 2 illustrates a depiction of a pill bottle with a dispenser, according to one or more embodiments;

FIGS. 3A-B illustrate another embodiment of a pill bottle and interior workings thereof, according to one or more embodiments;

FIG. 4 illustrates a system for determining compliance with prescription instructions, according to one or more embodiments;

FIGS. 5A-C are depictions of graphical user interfaces of a mobile application, according to one or more embodiments;

FIG. 6 illustrates a block diagram of a machine-learning module that may be used in any implementation of the disclosure, according to one or more embodiments;

FIG. 7A illustrates a flowchart of a method of secure pill dispensing, according to one or more embodiments;

FIG. 7B illustrates a flowchart of a method for assessing compliance with a prescription, according to one or more embodiments;

FIG. 7C illustrates a flowchart of a method for dispensing a pill using a rotating surface, according to one or more embodiments;

FIG. 8 is a block diagram of a computing system, according to one or more embodiments;

FIGS. 9A-B illustrate an embodiment of a pill bottle with a rotating disc, according to one or more embodiments.

FIG. 10A illustrates an exploded view of a pill container bottle with a dispenser opening, according to one or more embodiments.

FIG. 10B illustrates an isometric view of a pill bottle container with a dispenser opening, according to one or more embodiments.

FIG. 10C illustrates a side view of a pill bottle container with a dispenser opening, according to one or more embodiments.

FIG. 10D illustrates a top view of a revolving disc, according to one or more embodiments.

FIG. 10E illustrates a side view of a revolving disc, according to one or more embodiments.

FIG. 10F illustrates an isometric view of a revolving disc, according to one or more embodiments.

FIG. 10G illustrates a top view of a funnel, according to one or more embodiments.

FIG. 10H illustrates a first side view of a funnel, according to one or more embodiments.

FIG. 10I illustrates a second side view of a funnel, according to one or more embodiments.

FIG. 10J illustrates a third side view of a funnel, according to one or more embodiments.

FIG. 10K illustrates a side view of a bottom portion of a pill bottle container, according to one or more embodiments.

FIG. 10L illustrates a top view of a bottom portion of a pill bottle container, according to one or more embodiments.

FIG. 11 is a block diagram of bottle electronics for a pill bottle container, according to one or more embodiments.

### DETAILED DESCRIPTION

### Pill Dispensing System Overview

About 16.3 million people misuse prescriptions in a year. Economic cost of prescription opioid-related overdose, abuse, and dependence exceeded $1.5 trillion annually in 2020. Further, about 3 out of 10 adults have been addicted to opioids or have a family member who has been. 80% of patients receive opioids after low-risk surgery and 60% of people receiving 90 days of continuous opioid therapy remain on opioids years later. Aspects of the present disclosure may be used to reduce opioid abuse in the ongoing opioid epidemic. In an embodiment, aspects of the present disclosure can be used to track prescriptions relating to potentially addictive substances, such as barbiturates, buprenorphine, codeine, Adderall, fentanyl, hydrocodone, hydromorphone, methadone, morphine, oxycodone, oxymorphone, or tramadol. In some embodiments, aspects of the present disclosure can provide for ensuring compliance with prescription instructions, such as through machine-learning embodiments. Aspects of the present disclosure may provide for utilization of dispenser of pill bottles that may prevent a user from accessing medication of the pill bottles unauthorized.

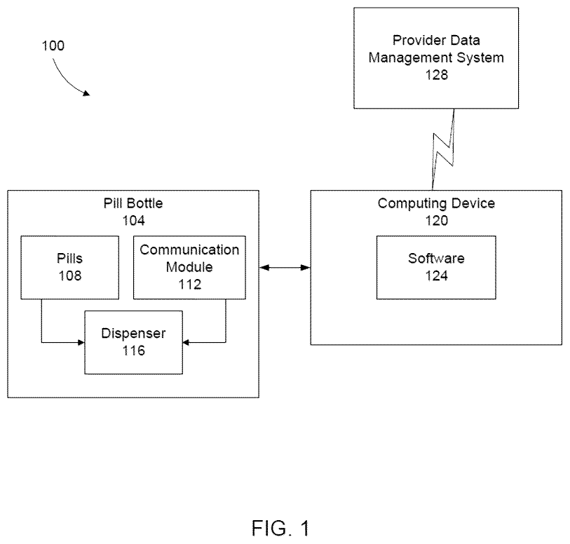

Referring now to FIG. 1, a block diagram of a system 100 for secure pill dispensing is shown. System 100 may include pill container or pill bottle 104. The pill container may be other types of containers besides a bottle, but a bottle is often referred to throughout as one example of a container. For example, the pill container may be a box, a sleeve, a compartment, a collection of compartments, a disk, a pouch, a package, a pop-top container, a squeeze open container, among others. FIG. 1 and many of the other figures use a pill bottle as an example, but it is understood that this pill bottle could take these other forms of pill containers.

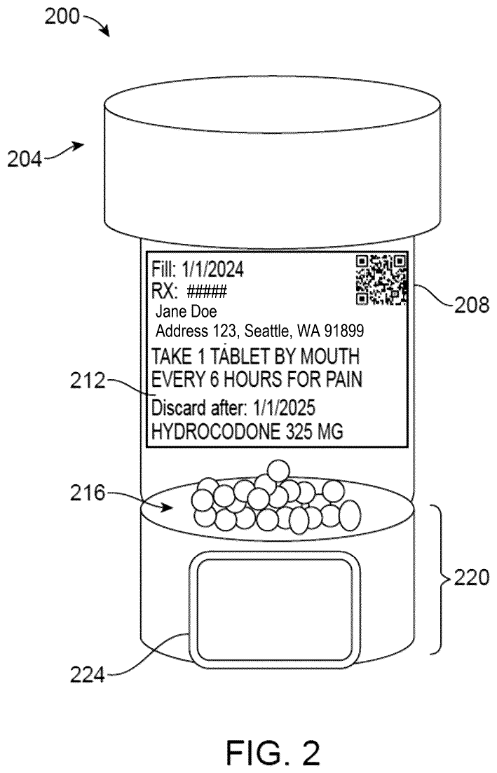

Pill bottle 104 in FIG. 1 and throughout the figures may be cylindrical, rectangular, round, or other shapes. Pill bottle 104 may include a bottom that may store one or more pills 108. Pills 108 may be round, circular, rectangular, and/or other shapes. Pills 108 may include tablets, capsules, or other forms of pills. Pills 108 may include, but is not limited to, barbiturates, buprenorphine, codeine, Adderall, fentanyl, hydrocodone, hydromorphone, methadone, morphine, oxycodone, oxymorphone, tramadol or other medications. Pill bottle 104 may include a top that may have a lid that secures pills 108 within the pill bottle 104. Pill bottle 104 may have an opaque surface that may prevent ultra-violet (UV) light from interacting with pill 108. Pill bottle 104 is described in greater detail below with reference to FIG. 2 and the other figures.

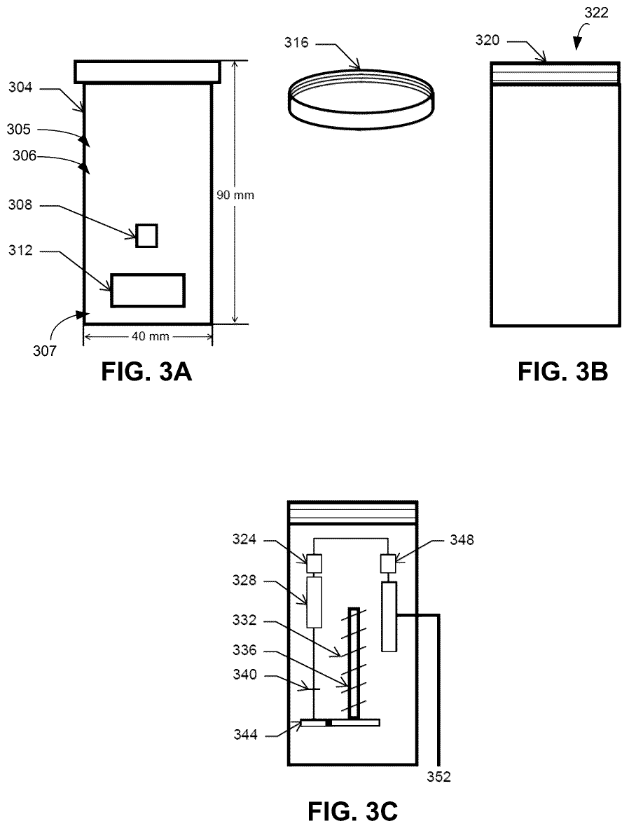

Pill bottle 104 may include communication module 112. A “communication module” as used in this disclosure is any device that is capable of transmitting and receiving electromagnetic signals. Communication module 112 may include, but is not limited to, Bluetooth, Near-Field communication (NFC), Wi-Fi, or other forms of communication. Pill bottle 104 may include a battery that may power communication module 112. Communication module 112 may be configured to communicate with one or more computing devices, such as, but not limited to, servers, smartphones, tablets, laptops, and/or other computing devices. In some embodiments, pill bottle 104 includes dispenser 116. A “dispenser” as used in this disclosure is a device capable of selectively releasing a medication. In some cases, the dispenser is referred to as an opening with a door, or a side structure opening when it is an opening in the side of the container structure. Dispenser 116 may include a door that may selectively open, releasing one or more pills 108. A door of dispenser 116 may be round, square, rectangular, or other shapes. Dispenser 116 may include one or more actuators that may open and/or close a door of dispenser 116. Dispenser 116 may be configured to dispense one or more pills 108 from pill bottle 104. In some embodiments, communication module 112 is electrically connected to dispenser 116 and/or a processor that is in communication with communication module 112 and/or dispenser 116. Dispenser 116 may be configured to activate based on data received from communication module 112. Dispenser 116 or in some cases called the side structure opening is described in further detail below with reference to FIG. 3B and other figures.

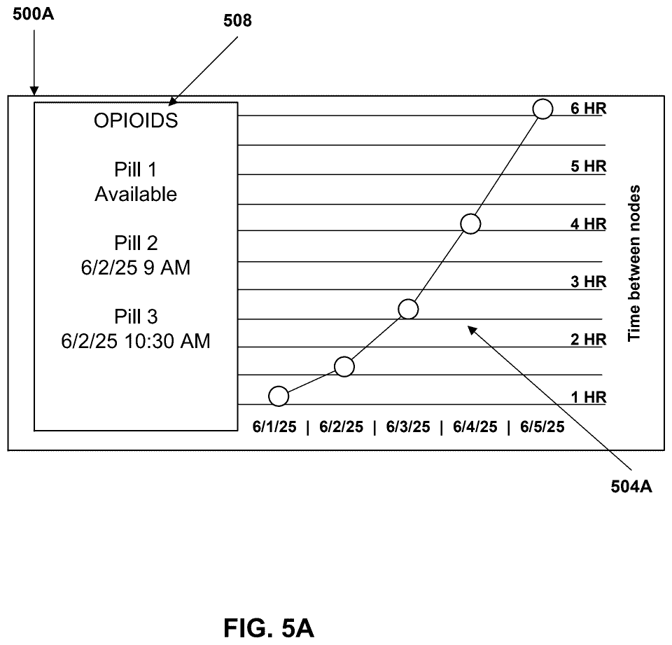

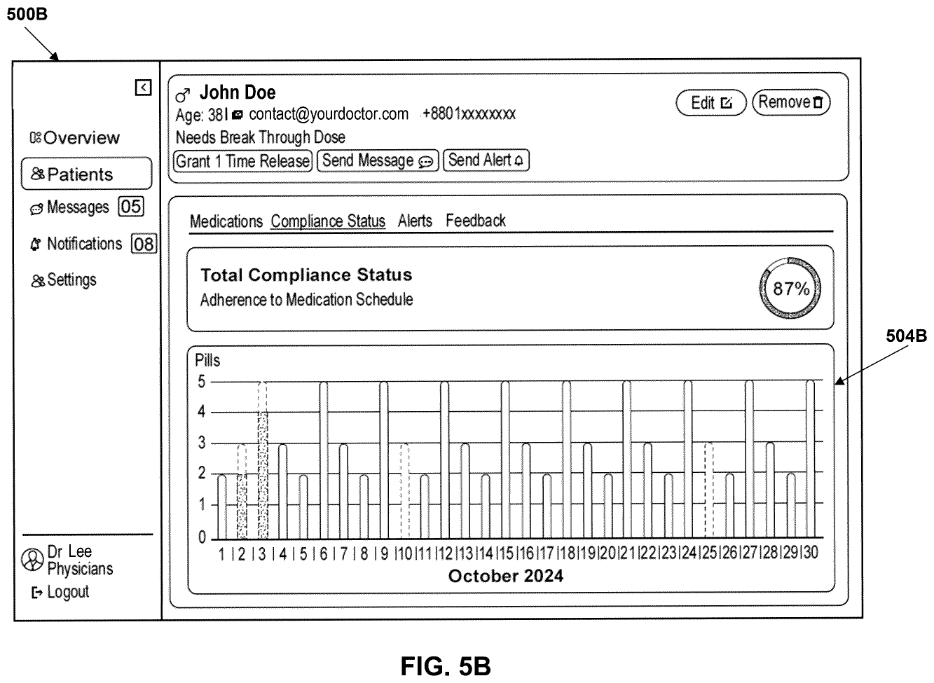

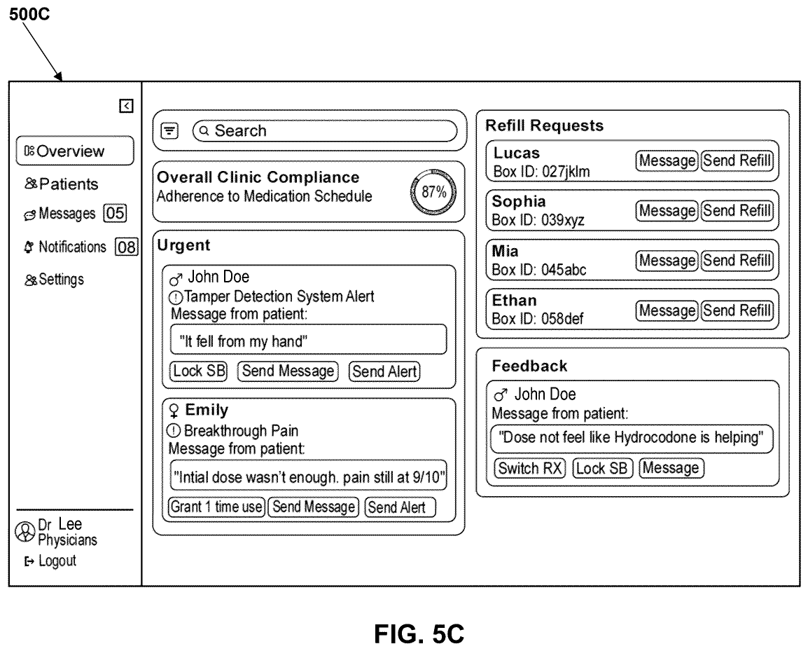

Referring still to FIG. 1, system 100 may include computing device 120. Computing device 120 may include, but is not limited to, a server, smartphone, laptop, tablet, or other device. Computing device 120 may have a transmitter and/or receiver that may be configured to communicate with pill bottle 104 through communication module 112, for instance over a Bluetooth, Wi-Fi, NFC, or other connection. Computing device 120 may be configured to run one or more software applications, such as software 124. Software 124 may run on the iOS, Android, or other mobile platforms. In some embodiments, software 124 is operable to run on Windows, MacOS, or other desktop/laptop platforms. Software 124 may be a web-portal application, mobile application, or other application. Software 124 may connect pill bottle 104 with computing device 120. In some embodiments, software 124 connects pill bottle 104 with a user profile and/or a provider data management system 128. A user profile of software 124 may include, but is not limited to, names, addresses, prescribers, prescriptions, geographical data, and/or other information. Software 124 may automatically generate a user profile of a user based on data received from provider management system 128. For instance, a user profile of provider management system 128 may be integrated into a user profile of software 124, which may automatically populate information such as, but not limited to, names, addresses, prescribers, prescriptions, medication types, geographical locations, and/or other information. In some embodiments, software 124 has a graphical user interface (GUI). A GUI of software 124 may allow a user to dispense one or more pills 108 through computing device 120, which may be in communication with pill bottle 104. A GUI of software 124 may be described in further detail below with reference to FIG. 5.

Software 124 may be embedded into a software of provider data management system 128. For instance, software 124 may operate alongside or within a software of provider data management system 128. Provider data management system 128 may be, but is not limited to, a pharmacy prescription system. Pharmacies provider data management (PDM) system 128 may be a part of include, but are not limited to, CVS, Walgreens, Walmart, or other pharmacies. Software 124 may be programmed to communicate with multiple pharmacies. For instance, software 124 may be programmed to synchronize listings of prescriptions for a user among two or more pharmacies. Software 124 may be programmed to track medication types, fill dates, quantities, prescriber data, and/or other information among two or more pharmacies. Software 124 may be programmed to provide a listing of prescription information to a user through a graphical user interface (GUI). For instance, software 124 may provide a user with information of which pharmacies filled which prescriptions, pick-up dates of different prescriptions of different pharmacies, and/or other information. Software 124 may track one or more prescriptions of one or more users. Software 124 may interact with two or more pharmacies PDM system's 128 to track and/or update multiple prescriptions of a single user, multiple prescriptions of multiple users, and/or other combinations of users.

As a non-limiting example, a user may have two prescriptions at a first pharmacy and a third prescription at a second pharmacy. The user may be provided a list through software 124 of which prescriptions are located where, with the first two prescriptions located at the first pharmacy and the third prescription located at the second pharmacy. Continuing this example, the user may be provided with information such as pick-up by dates, prescription quantities, total costs of all three prescriptions, and/or other information. Software 124 may provide an option to a user to fill and/or refill one or more prescriptions at a specific pharmacy. Software 124 may continually update information of a user's prescriptions through communication with multiple PDM systems 128 and may notify a user when a prescription is filled, when a prescription is ready, when a refill date is upcoming, and/or other information. In some embodiments, software 124 notifies a user that a prescription has the ability to be filled at a pharmacy that had been previously used by the user. As a non-limiting example, a user may have two prescriptions ready for pickup at a first location. Software 124 may prompt the user to fill a third prescription at the same location which may be more convenient for the user. In some embodiments, software 124 may communicate with one or more PDM systems 128 to locate and/or update inventory of one or more prescriptions. For instance and without limitation, various pharmacies may have various quantities of fentanyl or other drugs. Software 124 may communicate with multiple pharmacies PDM systems 128 to locate which pharmacies have a user's prescription in stock and may present these pharmacies to a user for selection through a GUI.

In some embodiments, locations of pharmacies with inventories of certain medications are communicated to a prescriber through software 124. A prescriber may select a pharmacy to fill a prescription at through software 124. In some embodiments, a prescriber writes prescription requests for patients through software 124. For instance, software 124 may be in communication with one or more PDM systems 128 which may allow a prescriber to send a prescription request directly to a pharmacy. Software 124 may provide information about a prescriber, prescriptions in progress, and/or other information to a user. In some embodiments, software 124 allows for a user to select a medication to be filled at a pharmacy and communicates this request to a prescriber through software 124. A prescriber may receive a request to fill a prescription through software 124 and may send a prescription to a PDM system 128 through software 124. Software 124 may provide a prescriber with information such as, but not limited to, last fill dates of a medication, pick-up dates of prescriptions, dose information of prescriptions, how long a prescription should last, and/or other information. Software 124 may allow for a prescriber to monitor one or more prescriptions of a user. For instance, software 124 may track a user's prescription activity to determine if the user is receiving multiple prescriptions of the same medication from various doctors, if the user is picking up their medication, if the user is following prescription instructions, or other determinations. In some embodiments, software 124 is programmed to compare two or more prescriptions for any potential chemical interactions of the two or more prescriptions. Software 124 may receive listings of interactions with various medications from one or more PDM system 128. If a potentially severe interaction between medications is determined, software 124 may alert PDM system 128, a prescriber, and/or the user of the potential interactions. In some embodiments, software 124 may classify prescriptions to one or more categories, such as, but not limited to, opioids, selective serotonin reuptake inhibitors (SSRI's), antipsychotics, benzodiazepines, antibacterials, or other categories. In some embodiments, software 124 classifies one or more prescriptions to categories of drug schedule drug listings provided by the United States Drug Enforcement Administration (DEA), such as, but not limited to, Schedule I, Schedule II, Schedule III, Schedule IV, and/or Schedule V drugs. Software 124 may utilize a classifier to classify prescriptions to one or more categories. A classifier may be trained with training data correlating prescriptions to categories of drugs. Training data may be received through user input, external computing devices, and/or previous iterations of processing. A classifier used by software 124 may be trained to classify prescriptions to categories of prescriptions, such as Schedule listings, antidepressants, SSRI's, opioids, antipsychotics, and/or other categories of drugs.

Still referring to FIG. 1, software 124 may notify a user when a prescription is in stock, when a prescription is ready for pick-up, when a prescription is being filled, and/or other information. In some embodiments, software 124 is able to learn user behavior and/or prescription patterns. Software 124 may determine which medications are routinely filled, which pharmacies are routinely used, which prescriptions are normally refiled, and/or other information. Based on previous user behavior, software 124 may provide recommendations of refills for prescriptions, recommendations of pharmacies, recommendations of dates to request prescriptions, and/or other recommendations. Software 124 may utilize a machine-learning algorithm that may be trained with training data correlating prescriptions to pharmacies. Training data may be received through user input, external computing devices, and/or previous iterations of processing. A machine-learning algorithm of software 124 may utilize user data continually to train and reinforce one or more machine-learning weights, parameters, and the like. For instance, and without limitation, a user may routinely fill a prescription at a first pharmacy around the 30th of each month. Software 124 may prompt a user to fill a prescription around the 30th of each month through a GUI, text, or other communication method. A user may receive a prescription that may be filled.

Software 124 may provide an optimized list of pharmacies from which a user may fill a prescription. For instance, software 124 may optimize pharmacies based on previously filled prescriptions, distances to a user, inventories of pharmacies, and/or other factors. Software 124 may utilize an optimization algorithm, such as, but not limited to, a continuous function optimization algorithm, a differentiable function algorithm, a bracketing algorithm, a local descent algorithm, and/or other algorithms. Optimization of pharmacy listings by software 124 may be based on distance to a user, pick-up dates of prescriptions, re-fill dates of prescriptions, or other factors. In some embodiments, optimization of pharmacy listings accounts for multiple prescriptions for multiple users. Optimization of multiple prescriptions of multiple users may aim to reduce the total time spent driving, reduce an amount of pharmacies needed to visit, and/or minimization of other factors. In some embodiments, a user inputs criteria for optimization to software 124. Software 124 may run an optimization algorithm to maximize or minimize one or more criterions received by user input.

PDM system 128 may receive a prescription from a medical provider. A prescription received from a medical provider may automatically be flagged for barbiturates, buprenorphine, codeine, Adderall, fentanyl, hydrocodone, hydromorphone, methadone, morphine, oxycodone, oxymorphone, or tramadol by software 124. A pharmacy of PDM system 128 may fill pill bottle 104 with one or more pills 108 based on a prescription received from a medical provider. Software 124 embedded into PDM system 128 may synchronize pill bottle 104 with a prescription. Synchronization may include matching or tagging pill bottle 104 to a prescription corresponding to pills 108. Pill bottle 104 may include an identifier. An “identifier” as used in this disclosure is a unique code that corresponds to a prescription. An identifier may be, but is not limited to, a quick-reference (QR) code, randomly generate strings, or other identifiers. Software 124 may be configured to manage and/or keep track of multiple pill bottles 104 through multiple identifiers that each may correspond to a prescription. Software 124 may provide PDM system 128 with listings of pill bottles 104. As a non-limiting example, software 124 may list pill bottle 104 as “Fentanyl Prescription” along with an identification number and/or string, a quantity of pills 108, a date pill bottle 104 was filled, an expected re-fill date, patient information, and/or other information. Synchronization of pill bottle 104 may allow for tracking of numerous pill bottles 104 that include various prescriptions. For instance, and without limitation, a PDM system 128 may be able to identify and track a plurality of pill bottles 104 through software 124 which may be distinct from other prescriptions and/or pill bottles within PDM system 128. For instance, pill bottles 104 may each have a unique identification number and/or string. In some embodiments, software 124 may provide real-time data to one or more providers, such as, but not limited to, current location of pill bottle 104, quantity of pills 108 remaining, type of medication of pills 108, time between doses of pills 108, and/or other information.

Referring still to FIG. 1, a user or patient may retrieve pill bottle 104 from a pharmacy of PDM system 128. A top of pill bottle 104 may be locked, preventing a user from accessing pills 108 of pill bottle 104. For instance, a top of pill bottle 104 may include a pre-threaded lid. An individual working at a pharmacy of PDM system 128 may apply a lock or locking lid to pill bottle 104 after filing pill bottle 104 with pills 108. In some embodiments, pill bottle 104 has a lid sensor that detects when a lid is applied to pill bottle 104. In some embodiments, when a lid is applied to pill bottle 104 indicating that pill bottle 104 is ready for pickup, a notification may be sent to PDM system 128 or computing device 120 through software 124 from a communication module of pill bottle 104. A sensor of pill bottle 104 may detect if a lid of pill bottle 104 is opened. An opening of a lid of pill bottle 104 may be unauthorized and may be determined to be an intrusion event. An “intrusion event” as used in this disclosure is any unauthorized access to pills 108 through pill bottle 104. An on-board memory of pill bottle 104 may record an instruction event, which may be communicated to a provider, user, or other entity through software 124. In some embodiments, sensor data is transmitted directly from pill bottle 104 to computing device 120, which determines an intrusion event.

A user may engage with software 124 through computing device 120, such as via a GUI of computing device 120. Computing device 120 may be a smartphone, tablet, laptop, or other device a user may be in possession of. Software 124 may provide one or more inputs a controller of pill bottle 104. Inputs may include, but are not limited to, a number of pills 108 to dispense, timing restrictions of dispensing pills 108, schedules of dispensing pills 108, maximum number of daily pills 108 to be dispensed, minimum time between dispensing pills 108, emergency overrides for dispensing a number of pills 108, and/or other inputs. A user may select an option to dispense a pill 108 from pill bottle 104 through software 124. In some embodiments, a prescriber or other individual activates software 124 to provide one or more inputs to a controller of pill bottle 104. In some embodiments, one or more inputs provided by software 124 are conveyed to a controller of pill bottle 104 through communication module 112. Software 124 may receive a request to dispense a pill 108 from computing device 120 and may compare the request to one or more thresholds. Thresholds may include, but are not limited to, times of day, quantity of pills 108, location of a user, time since last dose, time till next dose, last dose dispensing time, total quantity of pills 108 dispensed, and/or other thresholds. Thresholds may be set by a medical provider such as a doctor, nurse practitioner, or other medical provider. In some embodiments, thresholds may be automatically set by software 124 based on prescription information provided by provider data management system 128. For instance, based on a quantity of pills 108 and/or a type of medication of pills 108 software 124 may automatically set times of days for ingestion, quantities of pills 108 that may be ingested in a day, times between doses, and/or other threshold values. If a request from a user to dispense a pill 108 is determined to be acceptable by software 124, software 124 may communicate with pill bottle 104 via communication module 112 and/or computing device 120. Computing device 120 may send a command to pill bottle 104 to activate dispenser 116.

In some embodiments, a controller of pill bottle 104 and/or software 124 determines whether a pill 108 may be dispensed based on a prescription of the pill 108 and a last dispensing of a prior pill 108 communicated by the pill bottle. A last dispensing of a prior pill 108 may include, but is not limited to, a prior dispensing of a pill 108, one or more earlier pill 108 dispensings, or earlier to all prior pill 108 dispensings. Activation of dispenser 116 may cause one or more pills 108 to be released from pill bottle 104. In some embodiments, pill bottle 104 automatically dispenses one or more pills 108 based on dates, time of day, and/or other information. Software 124 may alert or remind a user that it's time to take a pill 108. Alerts may include texts, sounds, vibrations, push alerts through a GUI, or other alerts implemented via computing device 120.

In some embodiments, activation of dispenser 116 includes opening a door of dispenser 116 for a period of time. Periods of time may include, but are not limited to, 1 second, 2 seconds, 3 seconds, 4 seconds, 5 seconds, or times longer than 5 seconds. Periods of time may include ranges of time, such as but not limited to about 1-5 seconds, about 1-4 seconds, about 1-3 seconds, about 1-2 seconds, or any range within these ranges. In some embodiments, dispenser 116 includes a pill shaped holder. A pill shaped holder of dispenser 116 may allow for precise dispensing of pills 108, such as in increments of 1 pill. For instance, a pill shaped holder of dispenser 116 may be shaped to hold a single pill 108. A pill holder of dispenser 116 may be specific to a type of pill 108, such as in length and width. In other embodiments, a pill holder of dispenser 116 is shaped to universally hold various types of pills 108.

In some embodiments, pill bottle 104 includes one or more sensors. Sensors may include scales, accelerometers, or other sensors. One or more sensors of pill bottle 104 may be configured to determine how many pills 108 of pill bottle 104 were dispensed by dispenser 116. For instance, a weight sensor of pill bottle 104 may be calibrated to determine an exact weight of a total quantity of pills 108 and an individual or specific weight of a singular pill 108. Pill bottle 104 may include one or more processors and/or controllers that may be configured to receive data from communication module 112 and/or one or more sensors of pill bottle 104. In other embodiments, data from one or more sensors of pill bottle 104 and/or communication module 112 may be communicated to computing device 120, which may process any data remotely. Computing device 120 and/or a processor of pill bottle 104 may calculate quantities of pills 108 dispensed, remaining quantities of pills 108, activation times of dispenser 116, and/or other calculations. In some embodiments, pill bottle 104 is configured to count a number of times dispenser 116 is activated and communicate the number to computing device 120 and/or software 124. A processor of pill bottle 104 may generate a time stamp of when dispenser 116 was activated and may communicate the time stamp and corresponding activation of dispenser 116 to computing device 120.

In some embodiments, pill bottle 104 is configured to couple to or mate with a docking station. A “docking station” as used in this disclosure is a device that electrically connects with and communicates data to or from a pill bottle. A docking station may include a processor, memory, display device, or other computer components. In some embodiments, a docking station may include a charging and/or data port that may coupled to a charging and/or data port of pill bottle 104. A docking station may provide power to pill bottle 104. A docking station may receive data from an on-board memory of pill bottle 104. For instance, dispensing times, pill quantities, locational data, and/or other data may be transferred between on-board memory of pill bottle 104 and a processor of a docking station. A docking station may be configured to communicate with a doctor and/or PDM system 128, such as through software 124 or other communication methods.

Still referring to FIG. 1, a prescriber, such as a doctor, may provide an emergency override to pill bottle 104 through software 124. An emergency override may include activating dispenser 116 outside of normal dosage times, after already providing a dose to a user, and/or other situations. An emergency override may be recorded by software 124 and/or a memory of pill bottle 104. In some embodiments, an emergency override includes dispensing increased quantities of pills 108 from pill bottle 104, such as in the event of a breakthrough dose.

Computing device 120 and/or pill bottle 104 may determine a geographical location of a user and/or pill bottle 104. For instance, computing device 120 may determine pill bottle 104 is within a certain proximity to computing device 120 and is located at a same location as computing device 120. In some embodiments, pill bottle 104 includes a location tracker. A location tracker may include, but is not limited to, a global positioning system (GPS), ultra-wideband transmitters, IP address trackers, or other devices. A geo-location of a user and/or pill bottle 104 may be determined through global positioning systems (GPS), IP address tracking, crowd sourcing, and/or other forms of geo-location. Based on a location of a user and/or pill bottle 104, an on-board controller of pill bottle 104 may prevent activation of dispenser 115. In some embodiments, a geo-location is sent to computing device 120 through software 124. Computing device 120 may prevent or authorize an activation of dispenser 116 through one or more commands transmitted to pill bottle 104 through software 124. In some embodiments, geo-fencing is implemented by a locational tracker of pill bottle 104 and/or a location determination of computing device 120. For instance, within a certain geographical region that may be determined by a locational tracker of pill bottle 104, activation of dispenser 116 may be authorized by a controller of pill bottle 104 and/or software 124. Geographical regions may be set by a medical provider and/or by software 124 based on user information, such as, but not limited to, home addresses, work addresses, and the like. In some embodiments, pill bottle 104 includes an on-board processor and a GPS device. An on-board processor of pill bottle 104 may determine, based on a location of pill bottle 104, activation of dispenser 116 is either authorized or unauthorized. As a non-limiting example, pill bottle 104 may be geo-fenced within a house of a user. A user may activate dispenser 116 within their house, but if they leave their house and try to activate dispenser 116 an on-board processor of pill bottle 104 may prevent activation of dispenser 116 based on a geographical location of the user.

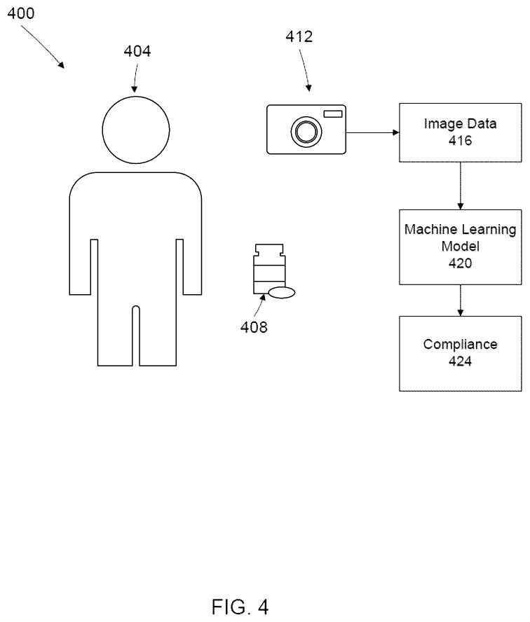

With continued reference to FIG. 1, software 124 may be configured to record a video of a user, such as through a camera of computing device 120. Videos may include, but are not limited to, 720p, 1080p, 4k, 60 fps, 120fps, and/or other video parameters. Video formats may include, but are not limited to, MP4, MOV, WMV, FLV, AVI, and/or other video formats. Computing device 120 may include one or more depth sensors, which may provide depth data to software 124. In some embodiments, videos are recorded and analyzed by software 124 in real-time. A user may start a video recording of themselves via software 124. In other embodiments, software 124 automatically starts recording video through computing device 120 based on activation of dispenser 116, which is communicated to computing device 120 through communication module 112. Software 124 may be configured to determine compliance with prescription instructions of pill 108 through a video recording of a user ingesting one or more pills 108. Compliance may include correctly ingesting an amount of pills 108 and non-compliance may include ingesting more or less than a correct amount of pills 108. Any data generated by software 124, computing device 120, and/or pill bottle 104 may be stored in a cloud-based storage system in communication with software 124, locally on computing device 120, or on an on-board memory of pill bottle 104. Data that may be stored by a cloud-based storage system, computing device 120, and/or pill bottle 104 may include, but is not limited to, locations of pill bottle 104, dispensing times, quantity of pills, number of times a dispenser of pill bottle 104 was activated, time in between doses, time until next dose, time since last dose, intrusion event details, prescriber information, user identities, battery status of pill bottle 104, and/or other information. In some embodiments, data generated by computing device 120, software 124, and/or pill bottle 104 may only be accessible only to authorized users. For instance, and without limitation, only medical providers, pharmacists, or other professionals may be authorized to access video recorded by computing device 120. In some embodiments, data generated by computing device 120, software 124, and/or pill bottle 104 is accessible to one or more users. Data may selectively be available to one or more users. For instance, users may be able to access data such as next dose time and quantity of pills remaining but may not be able to access intrusion event details, time in between doses, or other information. In some embodiments, data generated by pill bottle 104, computing device 120, and/or software 124 may be use in compliance reports, clinical trials, and/or other use cases. Any data generated by computing device 120, pill bottle 104, and/or software 124 may be used by a machine-learning model for training. Software 124 and/or computing device 120 may utilize a computer vision or other machine-learning model to analyze ingestion of one or more pills 108, such as described below with reference to FIG. 4.

Still referring to FIG. 1, software 124 and/or pill bottle 104 may send reminders to computing device 120 and/or may display reminders through a display of pill bottle 104. Reminders may include, but are not limited to, time for next dose, prescription refill dates, missed doses, and/or other reminders. Reminders may be sent to a user's computing device 120 and/or computing devices 120 of family members, medical providers, or other individuals in contact with a user. As a non-limiting example, a granddaughter may be sent a notification from software 124 and/or pill bottle 104 that it's time for her grandmother to take her medication. In some embodiments pill bottle 104 has an internal clock and/or timer and is configured to automatically send reminders for upcoming doses. In some embodiments, software 124 determines reminders for a user and communicates them to computing device 120. In some embodiments, software 124 is controlled and/or viewed by a third party. In other embodiments, software 124 is controlled and/or viewed by medical providers of a user.

### Pill Container Example

Referring now to FIG. 2, a pill bottle is illustrated. Pill bottle 200 may be the same as that of pill bottle as described above with reference to FIG. 1. Pill bottle 200 may be cylindrical, rectangular, hexagonal, octagonal, or other shapes. Pill bottle 200 may be any type of pill container, but a pill bottle is provided as an example. Pill bottle 200 may include a hollow structure 205 or outer shell or outer housing that includes one or more side surfaces 206, a top, and a bottom. The top may be open as a top structure opening that can receive pills and can be covered by a lid 204. The bottom may be open to form a bottom structure opening, or it may be closed. The bottom may include a base.

Pill bottle 200 may be a large-capacity pill bottle. A large-capacity pill bottle may be sized to store about 60 pills 216. Pill bottle 204 may be a small pill bottle. A small pill bottle 204 may be sized to store about 30 pills 216.

Pill bottle 200 may have an inner diameter of about 35 mm. In some embodiments, pill bottle 200 has a length of about 104 mm and a width of about 57 mm. Pill bottle 200 may have a length greater or less than about 104 mm and a width greater or less than about 57 mm.

In some embodiments, pill bottle 200 has an opaque surface. An opaque surface of pill bottle 200 may prevent unwanted chemical interactions between pills 216 and outside factors, such as UV light.

Pill bottle 200 may include lid 204, as noted above. Lid 204 may be a push-on, snap-on, twist-on or other type of lid. In some embodiments, lid 204 is tamper proof. For instance, lid 204 may be non-removable once applied to pill bottle 204, such as through an adhesive and/or locking mechanism. Lid 204 may prevent pills 216 from spilling out of pill bottle 200. In some embodiments, lid 204 may be colored, such as, but not limited to, red, yellow, blue, green, pink, and the like. A color of lid 204 may correspond to a specific medication of pills 216. As a non-limiting example, a red lid 204 may correspond to fentanyl, a yellow lid 204 may correspond to Adderall, and a blue lid 204 may correspond to barbiturates.

Pill bottle 200 may include identifier 208. Identifier 208 may be a quick response (QR) code. Identifier 208 of pill bottle 200 may identify a listing of pill bottle 200 in a provider data management system, such as provider data management system 128 as described above with reference to FIG. 1. In some embodiments, a user may scan identifier 208 with a computing device, such as a smartphone, which may cause the computing device to open up a mobile application, such as software 124 as described above with reference to FIG. 1. In some embodiments, a medical provider may scan identifier 128 with a computing device, which may cause the computing device to pull up a listing of pill bottle 200 in a provider data management system.

Identifier 208 may be an NFC chip, which may interact with one or more computing devices within a certain proximity, such as about 4 inches. Scanning an NFC chip of identifier 208 may cause an application to open up, show information of pill bottle 204, and/or other information.

Pill bottle 204 may include prescription information 212. Prescription information 212 may be an adhesive sticker that may be applied to a surface of pill bottle 200. Prescription information 212 may include information such as, but not limited to, fill date, discard date, RX numbers, patient name, pharmacy information, prescription instructions, discard after date, medication information, and/or other information.

Referring still to FIG. 2, pill bottle 200 may store one or more pills 216. Pills 216 may be large, small, tabular, or other shaped. Pill bottle 200 may store about 30, 60, 90, or other amounts of pills 216. Pills 216 may include barbiturates, buprenorphine, codeine, Adderall, fentanyl, hydrocodone, hydromorphone, methadone, morphine, oxycodone, oxymorphone, or tramadol and/or medications.

Pill bottle 200 may include dispensing compartment 220. Dispensing compartment 220 may be a bottom portion or a base of pill bottle 200 where pills 216 may be located. Dispensing compartment 220 may be shaped as a bottom of pill bottle 204. Dispensing compartment 220 may have one or more holes. One or more holes of dispensing compartment 220 may allow for one or more pills 216 to fall to dispenser 224 or a side structure opening. In some embodiments, dispensing compartment 220 may be motorized. For instance, a motor of dispensing compartment 220 may rotate a wheel or other component that may allow one or more pills 216 to fall into a bottom of the dispensing compartment 220 towards dispenser 224. As a non-limiting example, a motor of dispensing compartment 220 may rotate a wheel that may have a surface blocking one or more holes of dispensing compartment 220. Upon activation, a motor may rotate the wheel which may remove a surface of the wheel from the one or more holes of dispensing compartment 220 which may allow one or more pills 216 to fall towards dispenser 224. In some embodiments dispensing compartment 220 may include a door or slide that may allow one or more pills 216 to fall towards dispenser 224. A door or slide may be motorized. Motors of dispensing compartment 220 may include direct current (DC), alternating current (AC), brushless, or other motors. Dispensing compartment 220 may include a battery that may power dispenser 224 and/or one or more motors of dispensing compartment 220.

Pill bottle 200 may include an indicator. An “indicator” as used in this disclosure is a device that conveys information according to an even through visual elements, auditory elements, vibration elements, or a combination thereof. An indicator of pill bottle 200 may include a light emitting diode (LED) or other light source. An LED of pill bottle 200 may be in a form of an LED strip that may encompass a circumference of pill bottle 200. In some embodiments, an indicator of pill bottle 200 is a square, circular, or other shaped LED positioned near dispenser 224. An indicator of pill bottle 200 may be operable to display reg, green, blue, or any combination thereof. Various colors of an indicator may indicate different parameters to a user. For instance, a green color may indicate that pill bottle 200 is ready to dispense one or more pills 216. A yellow color may indicate that pill bottle 200 is waiting for confirmation to dispense one or more pills 216 from a mobile application. A red color may indicate that dispenser 224 is malfunctioning, there's an error in on-board memory of pill bottle 200, or other indications. An indicator of pill bottle 200 may animate. For instance, animations may include flashing, dimming, brightening, or other animations. In some embodiments, an indicator of pill bottle 200 is an LED strip that is operable to animate a singe LED at a time in a clockwise or counterclockwise direction. Animations of an indicator may occur based on various parameters of pill bottle 204. For instance, a flashing animation of an indicator may indicate to a user that it's time to take a pill 216. A brightening animation may indicate to a user that pill bottle 204 is dispensing pill 216 through dispenser 224. A dimming animation may indicate to a user that pill bottle 200 is done dispensing and is in a stand-by mode. An indicator may be turned off and not indicate anything to a user, such as in a stand-by mode.

An indicator may be one or more speakers, which may be configured to produce one or more sounds. Speakers of pill bottle 200 may emit sounds such as, but not limited to, chirps, chimes, beeps, rings, or other sounds. Various sounds emitted by one or more speakers of pill bottle 200 may correspond to various events. For instance, and without limitation, a chirp may indicate pill bottle 200 is turned on, a long beep may indicate a request for dispensing of pill 216 was sent, a low buzz may indicate dispenser 224 can't be activated at a certain time, or other combinations of sounds and indications.

An indicator may be a vibrational motor that may be configured to output one or more vibrations. Vibrations output by a vibrational motor may include, but are not limited to, short pulses, long vibrations, patterned vibrations, or other vibrations. Vibrations of a vibrational motor may correlate to one or more events. For instance, a short buzz may indicate pill bottle 200 is activate, a triple buzz may indicate a request to dispense pill 216 was successfully sent, a long buzz may indicate a reminder to take pill 216, or other vibrational outputs and events may be correlated.

In some embodiments, lid 204 is a smart lid. For instance, lid 204 may include one or more controllers, processors, sensors, memory, and the. In some embodiments, lid 204 has a dispenser that dispenses a pill from a top of pill bottle 200. A user may turn pill bottle 200 upside down, which may position one or more pills at a dispenser of lid 204. In some embodiments, pill bottle 200 includes one or more magnets or other components that cause electronic systems of pill bottle 200 to turn off if pill bottle 302004 is flipped upside down. For instance, in some embodiments, lid 204 includes an indicator, such as but not limited to a light emitting diode (LED) indicator, display screen, or other displaying element. Lid 204 may include a circular LED strip that encompasses a circumference of lid 204. A circular LED strip that encompasses a circumference of lid 204 may light various colors to indicate various events, such as, but not limited to, red to indicate locking of pill bottle 304, yellow to indicated dispensing of a pill, green to indicated a pill is ready to be dispense, or other combinations of colors corresponding to events. Lid 204 may unlock and/or lock itself to pill bottle 200, such as through a magnetic lock, based on one or more events. For instance, lid 204 may lock itself to pill bottle 304 outside of authorized dispensing times and may unlock itself during authorized dispensing times. Lid 204 may be rotatable. For instance, and without limitation, lid 316 may rotate around the top of pill bottle 200 while remaining locked to pill bottle 200. A user may rotate lid 204 until one or more “clicks” are produced, which may send a request to dispense a pill to a prescriber or pharmacy through software in communication with pill bottle 200 or to a controller of pill bottle 200. In some embodiments, rotating lid 204 to a number of clicks causes dispenser 224 to activate. A user may input a number of clicks to enter numbers of a password, such as a three-digit, four-digit, five-digit, or other-digit code through rotation of lid 204, which may have one or more numbers printed along a circumference of itself indicating which number lid 204 is currently positioned on.

Still referring to FIG. 2, pill bottle 200 may have one or more displays, such as digital screens LED screens, or other screen types. Displays of pill bottle 200 may be configured to display data received from software in communication with pill bottle 200, a controller of pill bottle 200, or both. Data may include, but is not limited to, quantity of pills remaining, time until next dose, time since last dose, count down until next dose, intrusion detection, or other information. Intrusion detection may include the status of a detected intrusion of a user trying to access 216 of pill bottle 200. A status of a detected instruction may include a no intrusion detected or intrusion detected status. Pill bottle 200 may have a digital screen that displays prescription information 212, dates, times, current dose, next scheduled dose, days until empty, last time a dose was taken, quantity of pills, communication status with a software, or other information. In some embodiments, pill bottle 200 has one or more LED lights that indicate various information. For instance, pill bottle 200 may have a first LED that may be positioned next to printed on text reading “ready”. The first LED may appear green, indicating that a pill is ready to be dispended or red indicating a pill is not ready to be dispensed. Pill bottle 200 may have a second LED located next to dispenser 224, which may flash green indicating dispenser 224 is dispensing properly or red indicating dispenser 224 is not working properly.

### Pill Container Example with Button

Referring now to FIG. 3A, another embodiment of pill bottle 304 is presented. Pill bottle 304 may be the same as that of pill bottle 104 as described above with reference to FIG. 1. Again, this may be any type of pill container, though a pill bottle is used as an example. The pill bottle embodiment of FIG. 3 may also be a hollow structure 305 that has one or more side surfaces 306, a top and bottom. The top may be open to form a top structure opening 322 (see FIG. 3B). The bottom 307 may be open to form a bottom structure opening, or it may be closed.

Pill bottle 304 may include button 308. Button 308 may be a clickable, capacitive, or other button. Button 308 may include a fingerprint scanner that may be configured to authenticate a user's identity while the user activates the button 308. Button 308 may be configured to send a signal to controller 324, which may activate dispenser 312. In some embodiments, button 308 is an emergency manual override. An emergency manual override may be implemented in scenarios in which a person prescribed a pill needs to take a pill outside of authorized dispensing times. Authorized dispensing times may include times and/or dates of scheduled doses. Unauthorized dispensing times may include times outside of normal dosing scheduling, dispensement outside a locational region, or other unauthorized dispensing times. As a non-limiting example, a user may need to take a breakthrough does for pain management. Button 308 may act as a manual emergency override that causes dispenser 312 to dispense a pill. An on-board memory of pill bottle 304 and/or software in communication with pill bottle 304 may record activation of button 308 and/or dispenser 312, such as times activated, time and date of activation, number of activation attempts, or other information. Dispenser 312 may be the same as that of dispenser 224 as described above with reference to FIG. 2. Pill bottle 304 may include threads 320. Threads 320 may be made of plastic or other materials. Threads 320 may have a clockwise or counterclockwise rotation. Threads 320 may be shaped to connect with one or more threads of lid 316. Lid 316 may include a pre-applied thread locker. A pre-applied thread locker may be an adhesive that may secure lid 316 to threads 320. In some embodiments, lid 316 may be secured to threads 320, which may prevent a user from accessing one or more medications in pill bottle 304. Lid 316 may be secured to threads 320 by a medical provider or other medical professional, such as a pharmacist.

In some embodiments, pill bottle 304 includes a speaker. A speaker may be piezoelectric, electromagnetic, or other type of speaker. A speaker of pill bottle 304 may be configured to produce a sound. Sounds include beeping, chirping, or any other sound. A speaker of pill bottle 304 may assist in a user finding pill bottle 304 when pill bottle 304 is lost. For instance, through software in communication with pill bottle 304, a user may request that pill bottle 304 plays a sound through a speaker to aid in fining pill bottle 304. In some embodiments, pill bottle 304 has a LED configured to flash a bright light that may assist in a user finding pill bottle 304 when pill bottle 304 is lost. A user may request through software in communication with pill bottle 304 aid in finding pill bottle 304 when pill bottle 304 is lost. Software may cause a speaker and/or LED light of pill bottle 304 to activate, which may cause pill bottle 304 to play a sound, emit a bright light, or combinations thereof. Software in communication with pill bottle 304 may display an indicator of pill bottle 304 through a display device of a computing device. For instance, an arrow, radius, or other graphical indicator may be displayed through a display device of a computing device in communication with software. In some embodiments, software may generate an augmented reality (AR) display that indicates where pill bottle 304 is located when lost. For instance, software may display real-time video of a surrounding of a computing device in communication with the software with a graphical icon superimposed in a three- dimensional mapping of a user's surroundings. Graphical images may include, but are not limited to, pills, bottles, or other symbols. A user may continually activate one or more speakers, lights, or other components of pill bottle 304 through a computing device in communication with software and pill bottle 304. In some embodiments, pill bottle 304 has a location device such as a GPS tag, ultra-band tag, or other device. Pill bottle 304 may communicate locational data of itself through a communication module based on locational data generated by a locational device. Software in communication with pill bottle 304 may determine a last known location of pill bottle 304 and may communicate the last known location of pill bottle 304 to a user through a computing device in communication with the software, which may aid a user in finding pill bottle 304 when pill bottle 304 is lost.

Referring now to FIG. 3B, an electrical schematic of an embodiment of pill bottle 304 is presented. Again, this may be any type of pill container, though a pill bottle is used as an example. Pill bottle 304 may include controller 324. Controller 324 may be a proportional integral (PI) controller, proportional derivative (PD) controller, proportional integral derivative (PID) controller, microcontroller, system on a chip (SoC), or other type of controller. Pill bottle 304 may include motor 328. Motor 328 may be an alternating current (AC) motor, direct current (DC) motor, or other motor. Controller 324 may be in electrical communication with motor 328, such as through one or more conductive wires. Motor 328 may be configured to move conveyer 332. Motor 328 may be in mechanical communication or connection with conveyer 332, directly or indirectly. For instance, a rotation of motor 328 may apply a torque to conveyor 332, which may cause conveyor 332 to rotate. Conveyer 332 may be any type of conveyer. In some embodiments, conveyer 332 is an auger style conveyer. Motor 328 may rotate conveyer 332 based on one or more commands received from controller 324. A rotation of conveyer 332 may allow for one or more pills to fall towards a bottom of pill bottle 304. In some embodiments, one or more wedges of conveyer 332 are sized to allow only a single pill to travel to a bottom of pill bottle 304. In other embodiments, one or more wedges of conveyer 332 are sized to allow multiple pills to travel to a bottom of pill bottle 304. A bottom of pill bottle 304 may include a spring door 336. Spring door 336 may be a one-way spring door. Spring door 336 may be configured to open in a single direction, with a spring providing opposing force against the single direction. Spring door 336 may be positioned near a bottom of conveyer 332 and/or pill bottle 304. For instance, spring door 336 may be positioned near dispenser 312 which may be located at a bottom of conveyer 332. Spring door 336 may move downwards due to mechanical force applied from conveyer 332, which may allow one or more pills to drop towards a bottom of pill bottle 304. Spring door 336 may oppose a downward force through a spring which may prevent one or more pills from accidentally falling to a bottom of pill bottle 304.

Pill bottle 304 may include a gear transfer 340 and gear set 344. Gear transfer 340 may transfer kinetic energy from motor 328 to gear set 344. Gear set 344 may be configured to rotate conveyer 332 based on kinetic energy received through gear transfer 340 and originating from motor 328. Pill bottle 304 may include power supply 348. Power supply 348 may be in electrical communication with controller 324 and/or motor 328. In some embodiments, power supply 348 acts as a battery and may be configured to provide voltage to controller 324 and/or motor 328. In some embodiments, power supply 348 is a battery, such as, but not limited to, a lithium-ion battery. Power supply 348 may be a ceramic, non-polarized, super, or other capacitor. Power supply 348 may be rechargeable. Pill bottle 304 may have a charging port that allows for power to be transferred from an external source to power supply 348.

In some embodiments, pill bottle 304 includes kinetic power generator 352. Kinetic power generator 352 may include, but is not limited to, a piezoelectric, electromagnetic, or electrostatic kinetic power generator. Kinetic power generator 352 may be configured to convert kinetic energy into electrical energy. For instance, a user may shake pill bottle 304 which may generate electrical energy via kinetic power generator 352. Energy converted by kinetic power generator 352 may be transferred to power supply 348, which may in turn power one or more components of pill bottle 304. Pill bottle 304 may include one or more sensors, such as a shock sensor. A shock sensor may include an accelerometer, gyroscope, or other motion sensor. A shock sensor of pill bottle 304 may determine excessive forces applied to pill bottle 304. In some embodiments, a shock sensor of pill bottle 304 may provide for intrusion detection of pill bottle 304. Intrusion detection may include detecting a user trying to access medication within pill bottle 304 without authorization. For instance, a user may throw pill bottle 304, attempt to twist off lid 316, or otherwise try to access medication of pill bottle 304. Controller 324 may be in electrical communication with a shock sensor and may deactivate motor 328 if an intrusion is detected, preventing a user from accessing medication of pill bottle 304. Pill bottle 304 may have a force sensor near dispenser 312, which may be configured to detect if a user attempts to try to open dispenser 312. One or more sensors of pill bottle 304 may be configured to detect an intrusion event and communicate the intrusion event to a computing device through a communication module or to controller 324. In some embodiments, pill bottle 304 includes a proximity sensor that detects a distance of itself in reference to lid 316. A proximity sensor may determine if lid 316 is closed or open. In some embodiments, a closing or opening of lid 316 may cause controller 324 to perform various processes. For instance, controller 324 may record a time and date that lid 316 was opened based on data received from a proximity sensor. Controller 324 may communicate an opening of lid 316 along with sensor data of a proximity sensor to one or more computing devices. Controller 324 may store a time and date that lid 316 was opened in an on-board memory of pill bottle 304. Controller 324 may determine pill bottle 304 is filled upon an activation of pill bottle 304 and a closing of lid 316. For instance, when a pharmacist fills pill bottle 304 and closes lid 316, controller 324 may determine through sensor data of a proximity sensor that pill bottle 304 is ready to be picked up or is “armed”.

### Compliance System

Referring now to FIG. 4, a system 400 for determining compliance of pill use is presented. System 400 may include user 404, pill bottle 408, and/or camera 412. Pill bottle 408 may be as described above with reference to FIGS. 3A-B. User 404 may be a patient or individual that has been prescribed a medication. Camera 412 may be a web-camera. In some embodiments, camera 412 may be part of a smartphone, tablet, laptop, or other device. Camera 412 may be activated by user 404. In other embodiments, a computing device in communication with camera 412 may automatically activate camera 412 based on one or more criteria, such as activation of a dispenser of pill bottle 408. Camera 412 may be configured to generate image data 416. Image data 416 may include, but is not limited to, colors, brightness, resolutions, depth data, and/or other data. For instance, in some embodiments, camera 412 includes a depth sensor that generates depth data. The image data 416 may include one or more photos or videos.

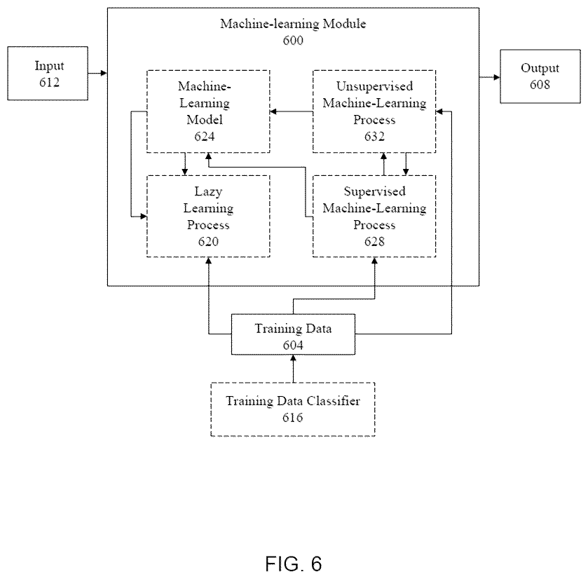

Image data 416 may be communicated to machine-learning model 420. machine-learning model 420 may be a computer vision or other type of machine-learning model. machine-learning model 420 may operate on a computing device of camera 412, such as a smartphone. In other embodiments, image data 416 may be communicated to a remote server that may be operating machine-learning model 420. machine-learning model 420 may be trained with training data correlating image data to compliances 424. Training data may be received through user input, external computing devices, and/or previous iterations of processing. Compliance 424 may be an indication that user 404 correctly took a right dose of medication from pill bottle 408 or that user 404 incorrectly took a dose of medication from pill bottle 408. machine-learning model 420 may be a computer vision model. A computer vision model may include a neural network that is trained to identify objects, people, events, and the like in images and/or videos. A computer vision model may utilize deep learning and/or other algorithms, such as described below with reference to FIG. 6.

machine-learning model 420 may be configured to identify three-dimensional (3D) spaces in a cartesian coordinate system (x,y,z). For instance, machine-learning model 420 may be trained or programmed to identify a location of user 404 and/or pill bottle 408 in a 3D space. machine-learning model 420 may be trained to identify pill bottle 408 separately from user 404. For instance, machine-learning model 420 may be trained to track locations and/or movements of both user 404 and/or pill bottle 408. machine-learning model 420 may be trained to identify one or more actions of user 404. For instance, machine-learning model 420 may identify walking, talking, and/or other actions. In some embodiments, machine-learning model 420 may identify an action of user 404 receiving a pill from pill bottle 408. machine-learning model 420 may identify an action of user 404 swallowing or otherwise ingesting a pill. In some embodiments, machine-learning model 420 may identify individual pills and may track individual pills throughout a video recording. For instance, machine-learning model 420 may be trained to identify a pill is in user's 404 hand, mouth, or other location. Based on a tracking of user 404′s actions, machine-learning model 420 may determine or assess compliance 424 of user 404 and provide an output related to compliance 424. For instance, machine-learning model 420 may output that a user 404 has high compliance 424, medium compliance 424, low compliance 424, or levels in between. High compliance 424 may include user 404 correctly swallowing a correct dose of medication from pill bottle 408. Medium compliance 424 may include user 404 ingesting a pill from pill bottle 408 but then requesting additional unauthorized doses. A low compliance 424 may include user 404 not swallowing any dose from pill bottle 408 or swallowing more than a prescribed dose of medication from pill bottle 408. In an event low compliance 424 is determined, an alert or notification may be sent to a medical provider or other professional. Outputs of machine-learning model 420 may be sent to a computing device that may update information such as, but not limited to, time since last dose, quantity of pills remaining, time until next dose, and/or other information. A computing device in communication with machine-learning model 420 may keep track of user 404′s progress of ingesting does of medication from pill bottle 408. In some embodiments, machine-learning model 420 acts in real-time. In other embodiments, machine-learning model 420 analyzes a pre-recorded video.

machine-learning model 420 may provide various assessments and/or details related to compliance 424. For instance, machine-learning model 420 may provide outputs such as, but not limited to, a total time it took for user 404 to ingest a pill from pill bottle 408, a background location of where user 404 ingested a pill from pill bottle 408, if low compliance 424 is determined due to a malfunctioning of pill bottle 408, if more than a single dose is released from pill bottle 408, emergency override scenarios, time of day ingestion took place, or other assessments. Outputs and/or assessments of machine-learning model 420 may be reported to one or more medical providers, third parties, and/or user 404. A video recorded by camera 412 may be accessible to software of a computing device, such as software 124 as described above with reference to FIG. 1. In some embodiments, a video recorded by camera 412 may be stored locally on a computing device or may be sent to cloud-storage.

In some embodiments, machine-learning model 420 may be trained to verify an identify of user 404. For instance, user 404 may attempt to have someone other than a person for whom a prescription is assigned take a dose from pill bottle 408. machine-learning model may be trained with training data to identify a user based on, but not limited to, facial structures, body mapping, general cadence, or other information. machine-learning model 420 may output a determination that user 404 is authorized, unauthorized, or that machine-learning model 420 is unsure if it's really user 404 or not. Outputs of machine-learning model 420 pertaining to an identify of user 404 may be communicated to software such as software 124 described above, which may take one or more actions based on one or more assessments provide by machine-learning model 420. For instance, upon authorization of user 404, software may activate dispensment of a pill from pill bottle 408. Upon detection of an unauthorized person attempting to take a dose from pill bottle 408, software may contact medical providers, user 404, pharmacies, local authorities, or other entities. If machine-learning model 420 provides an output of unsureness of an authorization of user 404, software may provide additional options of authorization to user 404, such as eye scans, fingerprint identification, two-factor authentication, or other forms of verification.

### Graphical User Interface Examples

Referring now to FIG. 5A, a graphical user interface (GUI) 500A is presented. GUI 500 may be displayed on a screen of, but not limited to, a monitor, smartphone, tablet, laptop, and/or other device. GUI 500A may include one or more graphical icons. Graphical icons ay correspond to one or more event handlers. Event handlers of GUI 500A may perform a computer action upon interaction with one or more graphical icons of GUI 500A. For instance, GUI 500A may have a graphic icon that may have an event handler that displays a graph upon a user clicking, tapping, or otherwise interacting with the graph icon. GUI 500A may include graph 504A. Graph 504A may be a depiction of a time in-between doses of a medication of a user. Graph 504A may be a dotted, lined, or other graph. Graph 504A may have a y-axis of hours and an x-axis of dates. Graph 504A may represent an increase or decrease in time between does over a period of time. A user may filter through various medications, dates, and/or other information through GUI 500A, which may cause graph 504A to update. In some embodiments, GUI 500A includes pill information 508. Pill information 508 may include a listing of pills and an indication of an availability of each pill in the listing of pills. For instance, a first pill may be currently available for ingestion, a second pill may be ready at 9 AM, a third pill may be ready by 10:30 AM, and a fourth pill may be ready by 12 PM. A user may interact with pill information 108, such as selecting one or more listing of pills. Selection of one or more pills may cause GUI 500A to display more details of one or more pills, such as medication type, pill name, Rx numbers, and/or other information. In some embodiments, GUI 500A may display a likelihood of addiction to one or more medications. For instance, a computing device in communication with GUI 500A may determine based on frequency of ingestion that a user may either have a high likelihood of becoming addicted to a medication or a low likelihood of a user becoming addicted to a medication. GUI 500A may display a likelihood of addiction per each medication type. A likelihood of addition may be represented by a percentage value, a value out of 5, a value out of 10, a number of stars, or other representations.

Referring now to FIG. 5B, a graphical user interface (GUI) 500B is presented. GUI 500 may be displayed on a screen of, but not limited to, a monitor, smartphone, tablet, laptop, and/or other device. GUI 500B may include one or more graphical icons. Graphical icons ay correspond to one or more event handlers. Event handlers of GUI 500B may perform a computer action upon interaction with one or more graphical icons of GUI 500B. For instance, GUI 500B may have a graphic icon that may have an event handler that displays a graph upon a user clicking, tapping, or otherwise interacting with the graph icon. GUI 500B may include graph 504B. Graph 504B may be a depiction of a total compliance status of a user. Graph 504B may be a bar chart, pie chart, or other graph. Graph 504B may have a y-axis of pills and an x-axis of dates. A user may filter through various medications, dates, and/or other information through GUI 500B, which may cause graph 504B to update. In some embodiments, GUI 500B includes compliance information, such as instances when a user did or did not take a pill when directed to, alerts the user did or did not respond to, feedback about the user's compliance, and the like. GUI 500B may be presented to a medical professional such that the medical professional can view the user's compliance with one or more prescriptions. GUI 500B may also include interactive elements that allow a medical professional to grant additional doses for a prescription, sends a message to the user, send an alert to a family member of the user, and the like.

Referring now to FIG. 5C, a graphical user interface (GUI) 500C is presented. GUI 500 may be displayed on a screen of, but not limited to, a monitor, smartphone, tablet, laptop, and/or other device. GUI 500C may include one or more graphical icons. Graphical icons ay correspond to one or more event handlers. Event handlers of GUI 500C may perform a computer action upon interaction with one or more graphical icons of GUI 500C. For instance, GUI 500C may have a graphic icon that may have an event handler that displays user information for users that see a medical professional being presented with the GUI 500C. User information may include overall compliance with prescriptions associated with a user, alerts associated with a user, refill requests, feedback about medication side effects from users, and the like.

### Machine-Learning Module

Referring to FIG. 6, an exemplary machine-learning module 600 may perform machine-learning process(es) and may be configured to perform various determinations, calculations, processes and the like as described in this disclosure using a machine-learning process. Machine-learning module 600 may utilize training data 604. For instance, and without limitation, training data 604 may include a plurality of data entries, each entry representing a set of data elements that were recorded, received, and/or generated together. Training data 604 may include data elements that may be correlated by shared existence in a given data entry, by proximity in a given data entry, or the like. Multiple data entries in training data 604 may demonstrate one or more trends in correlations between categories of data elements. For instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related to training data 604 according to various correlations. Correlations may indicate causative and/or predictive links between categories of data elements, which may be modeled as relationships such as mathematical relationships by machine-learning processes as described in further detail below. Training data 604 may be formatted and/or organized by categories of data elements. Training data 604 may, for instance, be organized by associating data elements with one or more descriptors corresponding to categories of data elements. As a non-limiting example, training data 604 may include data entered in standardized forms by one or more individuals, such that entry of a given data element in a given field in a form may be mapped to one or more descriptors of categories. Elements in training data 604 may be linked to descriptors of categories by tags, tokens, or other data elements. Training data 604 may be provided in fixed-length formats, formats linking positions of data to categories such as comma-separated value (CSV) formats and/or self-describing formats. Self-describing formats may include, without limitation, extensible markup language (XML), JavaScript Object Notation (JSON), or the like, which may enable processes or devices to detect categories of data.

With continued reference to FIG. 6, training data 604 may include one or more elements that are not categorized. Uncategorized data of training data 604 may include data that may not be formatted or containing descriptors for some elements of data. In some embodiments, machine-learning algorithms and/or other processes may sort training data 604 according to one or more categorizations. Machine-learning algorithms may sort training data 604 using, for instance, natural language processing algorithms, tokenization, detection of correlated values in raw data and the like. In some embodiments, categories of training data 604 may be generated using correlation and/or other processing algorithms. As a non-limiting example, in a body of text, phrases making up a number “n” of compound words, such as nouns modified by other nouns, may be identified according to a statistically significant prevalence of n-grams containing such words in a particular order. For instance, an n-gram may be categorized as an element of language such as a “word” to be tracked similarly to single words, which may generate a new category as a result of statistical analysis. In a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 604 to be made applicable for two or more distinct machine-learning algorithms as described in further detail below. Training data 604 used by machine-learning module 600 may correlate any input data as described in this disclosure to any output data as described in this disclosure, without limitation.

Further referring to FIG. 6, training data 604 may be filtered, sorted, and/or selected using one or more supervised and/or unsupervised machine-learning processes and/or models as described in further detail below. In some embodiments, training data 604 may be classified using training data classifier 616. Training data classifier 616 may include a classifier. A “classifier” as used in this disclosure is a machine-learning model that sorts inputs into one or more categories. Training data classifier 616 may utilize a mathematical model, neural net, or program generated by a machine-learning algorithm. A machine-learning algorithm of training data classifier 616 may include a classification algorithm. A “classification algorithm” as used in this disclosure is one or more computer processes that generate a classifier from training data. A classification algorithm may sort input into categories and/or bins of data. A classification algorithm may output categories of data and/or labels associated with the data. A classifier may be configured to output a datum that labels or otherwise identifies a set of data that may be clustered together. Machine-learning module 600 may generate a classifier, such as training data classifier 616 using a classification algorithm. Classification may be performed using, without limitation, linear classifiers such as without limitation logistic regression and/or naive Bayes classifiers, nearest neighbor classifiers such ask-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 616 may classify elements of image data to one compliance.

Still referring to FIG. 6, machine-learning module 600 may be configured to perform a lazy-learning process 620 which may include a “lazy loading” or “call-when-needed” process and/or protocol. A “lazy-learning process” may include a process in which machine-learning is performed upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 604. Heuristics may include selecting some number of highest-ranking associations and/or training data 604 elements. Lazy learning may implement any suitable lazy learning algorithm, including without limitation a K-nearest neighbors algorithm, a lazy naive Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Still referring to FIG. 6, machine-learning processes as described in this disclosure may be used to generate machine-learning models 624. A “machine-learning model” as used in this disclosure is a mathematical and/or algorithmic representation of a relationship between inputs and outputs, as generated using any machine-learning process including without limitation any process as described above and stored in memory. For instance, an input may be sent to machine-learning model 624, which once created, may generate an output as a function of a relationship that was derived. For instance, and without limitation, a linear regression model, generated using a linear regression algorithm, may compute a linear combination of input data using coefficients derived during machine-learning processes to calculate an output. As a further non-limiting example, machine-learning model 624 may be generated by creating an artificial neural network, such as a convolutional neural network comprising an input layer of nodes, one or more intermediate layers, and an output layer of nodes. Connections between nodes may be created via the process of “training” the network, in which elements from a training data 604 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 6, machine-learning algorithms may include supervised machine-learning process 628. A “supervised machine-learning process” as used in this disclosure is one or more algorithms that receive labelled input data and generate outputs according to the labelled input data. For instance, supervised machine-learning process 628 may include images as described above as inputs, cropped faces of images as outputs, and a scoring function representing a desired form of relationship to be detected between inputs and outputs. A scoring function may maximize the probability that a given input and/or combination of elements inputs is associated with a given output to minimize a probability that a given input is not associated with a given output. A scoring function may be expressed as a risk function representing an “expected loss” of an algorithm relating inputs to outputs, where loss is computed as an error function representing a degree to which a prediction generated by the relation is incorrect when compared to a given input-output pair provided in training data 604. Persons skilled in the art, upon reviewing the entirety of this disclosure, will be aware of various possible variations of at least a supervised machine-learning process 628 that may be used to determine relation between inputs and outputs. Supervised machine-learning processes may include classification algorithms as defined above.

Further referring to FIG. 6, machine-learning processes may include unsupervised machine-learning processes 632. An “unsupervised machine-learning process” as used in this disclosure is a process that calculates relationships in one or more datasets without labelled training data. Unsupervised machine-learning process 632 may be free to discover any structure, relationship, and/or correlation provided in training data 604. Unsupervised machine-learning process 632 may not require a response variable. Unsupervised machine-learning process 632 may calculate patterns, inferences, correlations, and the like between two or more variables of training data 604. In some embodiments, unsupervised machine-learning process 632 may determine a degree of correlation between two or more elements of training data 604.

Still referring to FIG. 6, machine-learning module 600 may be designed and configured to create a machine-learning model 624 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of I divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 6, machine-learning algorithms may include, without limitation, linear discriminant analysis. Machine-learning algorithms may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naive Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

In some embodiments, a machine-learning module 600 trains the machine-learning model 624 based on a set of training examples in the training data 604. Each training example includes input data to which the machine-learning model is applied to generate an output. For example, each training example may include user data, compliance information, prescription information, and the like. In some cases, the training examples also include a label which represents an expected output of the machine-learning model. In these cases, the machine-learning model is trained by comparing its output from the input data of a training example to the label for the training example. In general, during training with labeled data, the set of parameters of the model may be set or adjusted to reduce the difference between the output for the training example (given the current parameters of the model) and the label for the training example.

The machine-learning module 600 may apply an iterative process to train the machine-learning model 624 whereby the machine-learning module 600 updates parameter values of the machine-learning model 624 based on each of the set of training examples. The training examples may be processed together, individually, or in batches. To train the machine-learning model 624 based on a training example, the machine-learning module 600 applies the machine-learning model 624 to the input data in the training example to generate an output based on a current set of parameter values. The machine-learning module 600 scores the output from the machine-learning model using a loss function. A loss function is a function that generates a score for the output of the machine-learning model such that the score is higher when the machine-learning model 624 performs poorly and lower when the machine-learning model 624 performs well. In cases where the training example includes a label, the loss function is also based on the label for the training example. Some example loss functions include the mean square error function, the mean absolute error, hinge loss function, and the cross-entropy loss function. The machine-learning module 600 updates the set of parameters for the machine-learning model 624 based on the score generated by the loss function. For example, the machine-learning module 600 may apply gradient descent to update the set of parameters.

In some embodiments, the machine-learning module 600 may retrain the machine-learning model 624 based on the actual performance of the model after the model has been deployed to provide service to users. For example, if the machine-learning model is used to predict a likelihood of an outcome of an event, the machine-learning module 600 may log the prediction and an observation of the actual outcome of the event. Alternatively, if the machine-learning model 624 is used to classify an object, the machine-learning module 600 may log the classification as well as a label indicating a correct classification of the object (e.g., following a human labeler or other inferred indication of the correct classification). After sufficient additional training data has been acquired, the machine-learning training module 600 re-trains the machine-learning model 624 using the additional training data, using any of the methods described above. This deployment and re-training process may be repeated over the lifetime use for the machine-learning model 624. This way, the machine-learning model 624 continues to improve its output and adapts to changes in the system environment, thereby improving the functionality of the machine-learning module 600 as a whole in its performance of the tasks described herein.

### Method of Pill Dispensing

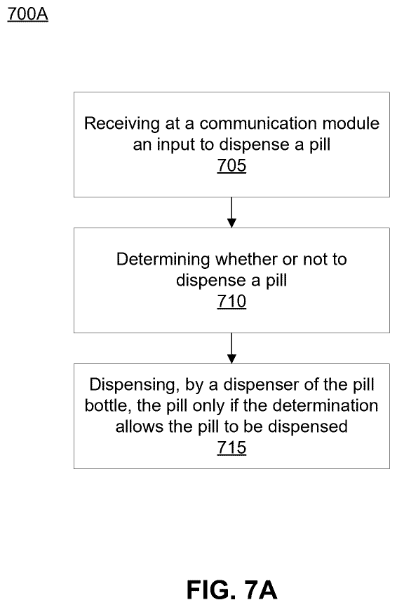

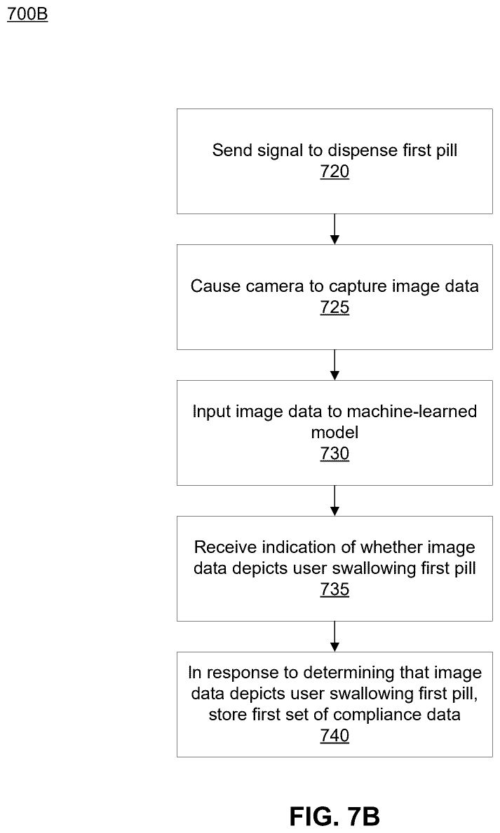

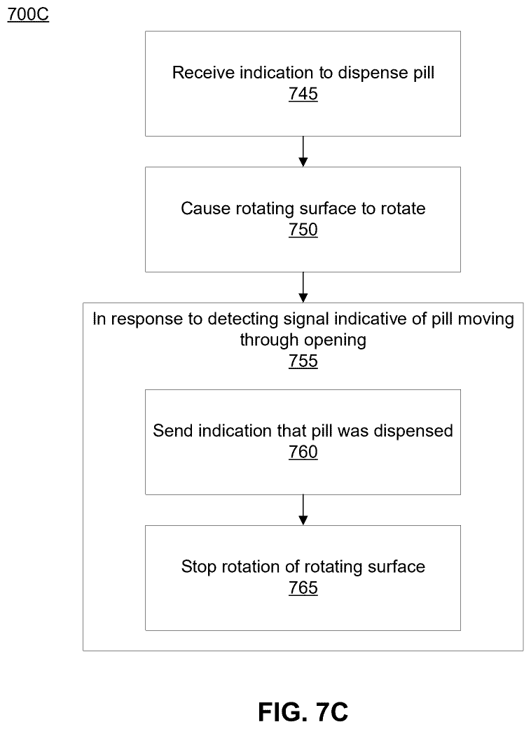

Referring now to FIG. 7A, a method 700A of secure pill dispensing is presented. This is just one example of this method that may be performed. In embodiments, there may be more, fewer, or different steps included in the method.

At step 705, method 700A includes receiving, at a communication module of a pill bottle, input to dispense a pill. Input may be received from a user, a prescriber, a family member of a user, or other individual. Input may be received through software in communication with the pill bottle or directly through one or more buttons of the pill bottle. Input may include, but is not limited to, requests for a dose, schedules of doses, next does time, last dose time, time in between doses, total quantity of doses per day, quantity of pills, and/or other inputs. This step may be implemented, without limitation, as described above with reference to FIGS. 1-6.

At step 710, method 700A includes determining whether or not to dispense the pill. A controller of the pill bottle or software in communication with the pill bottle may determine whether or not to dispense the pill. A determination of whether or not to dispense the pill may be based on, but is not limited to, prescription instructions corresponding to the pill, a time since a last dose was administered, location of the pill bottle, time in between doses, time until next dose, and/or other information. In some embodiments, authorization of a user may be factored into a determination of whether or not to dispense the pill. For instance, a user may not be authorized as determined by a machine-learning model, which may prevent a pill from dispensing. This step may be implemented, without limitation, as described above with reference to FIGS. 1-6.

At step 715, method 700 includes dispensing, by a dispenser of the pill bottle, the pill only if the determination allows the pill to be dispensed. A dispenser of a pill bottle may include a spring door and/or conveyor. In some embodiments, a motor and/or conveyor of a pill bottle may work together to dispense a pill. This step may be implemented, without limitation, as described above with reference to FIGS. 1-6.

### Method of Compliance Assessment

Referring now to FIG. 7B, a method 700B of assessing compliance with a prescription is presented. . . . This is just one example of this method that may be performed. In embodiments, there may be more, fewer, or different steps included in the method.

A controller of the pill bottle or software in communication with the pill bottle may determine whether or not a user is in compliance with their prescription. The controller may be in communication with a client device of the user. In some embodiments, the client device sends a signal to the controller at the pill bottle to dispense a first pill. The client device causes a camera to capture image data. The client device captures image data and inputs the image data into a machine-learned model that is trained to determine whether the image data depicts the user associated with the pill bottle swallowing the first pill. The client device receives, from the machine-learned model, an indication of whether the image data depicts the user swallowing the first pill. In response to determining that the image data depicts the user swallowing the first pill, the client device or controller stores a first set of compliance data in association with an identifier of the user. The first set of compliance data indicates that the user swallowed the first pill and may be displayed at a GUI such as that described in relation to FIG. 5B.

The method 700B may include additional or alternative steps to those shown in FIG. 7B. In some embodiments, the client device determines that the current time is in a time window for dispensing a second pill. In response to determining that a most recently stored set of compliance data is indicative of the user swallowing a pill, the client device sends a second signal to the controller to dispense a second pill.

In some embodiments, in response to determining that the image data does not depict the user swallowing the first pill, the client device sends an alert to a second client device associated with a medical entity, family member, pharmacy, or the like. The client device stores a second set of compliance data in association with the identifier of the user, where the second set of compliance data indicates that the user did not swallow the first pill.

In some embodiments, the client device determines that the current time at the client device is in a time window for dispensing a second pill. In response to determining that a most recently stored set of compliance data indicates that the user did not swallow a pill, the client device sends an alert to a medical entity associated with the user.

In some embodiments, the indication of whether the image data depicts the user swallowing the pill is an indication of a likelihood that the user swallowed the first pill. The client device may trigger an alert in response to the likelihood of being outside a threshold tolerance. In some embodiments, a first class of pills are associated with a lower threshold tolerance than a second class of pills.

In some embodiments, machine-learned model is trained on a first set of labeled image data that depicts one or more of a set of throat movements. This image data is labeled as depicting swallowing, and image data not depicting one or more of the set of throat movements is labeled as not depicting swallowing. The client device or controller may train the machine-learned model on a second set of labeled image data, which includes image data of the user labeled as depicting the user and image data that is not of the user labeled as not depicting the user.

In some embodiments, the client device receives, via a user interface, an indication that the user lost the pill bottle and sends an indication that the user lost the pill bottle to a medical entity associated with the user.

In some embodiments, the client device receives, via a user interface, an indication that the user lost the pill bottle. The client device requests, from the controller at the pill bottle, a current location of the pill bottle. The client device compares the current location of the pill bottle to the current location of the client device. In response to the current location of the pill bottle being outside of a threshold distance from the current location of the client device, the client device sends a request for new pills to a second client device associated with a medical entity.

Method of Pill Dispensing

Referring now to FIG. 7C, a method 700C of dispensing a pill using a rotating surface is presented. A controller of the pill bottle or software in communication with the pill bottle may be used to control the rotating surface. The controller may be in communication with a client device of the user. In some embodiments, the controller at the pill bottle receives an indication to dispense a pill. The pill bottle contains at least one pill in a first enclosure and a first pill is positioned above a rotating surface within the pill bottle. The controller causes the rotating surface within the pill bottle to rotate. The rotating surface includes a plurality of slots with at least one slot wide enough for the pill to move through the slot, and an opening is below the rotating surface in the pill bottle. In response to detecting, via a sensor configured at an opening of the pill bottle, a signal indicative of a pill moving through the opening, the controller sends an indication that the pill was dispensed to a client device and stops rotation of the rotating surface.

The method 700C may include additional or alternative steps to those shown in FIG. 7C. In some embodiments, each slot of the plurality of slots has a different width than the other slots, such that pills of different sizes of fit through different slots.

In some embodiments, the controller sends a notification that the pill was dispensed at the pill bottle. The notification is integrated into an electronic record for a user that is stored in an electronic record system associated with a medical entity.

In some embodiments, in response to determining that a user associated with the pill bottle is not in compliance with one or more medication management rules, the controller causes the rotating surface to lock, which prevents the rotating surface from rotating until a notification from a medical entity associated with a user of the client device is received.

In some embodiments, in response to detecting the signal indicative of a pill moving through the opening, the controller increases a count of pills dispensed at the pill bottle. In response to determining that that count has exceeded a threshold, the controller locks the rotating surface such that the rotating surface is prevented from rotating until a notification from a medical entity associated with a user of the pill bottle is received.

In some embodiments, in response to determining that the count has exceeded a threshold, the controller sends an alert that includes the count to a client device of a medical entity.

### Computer Diagram

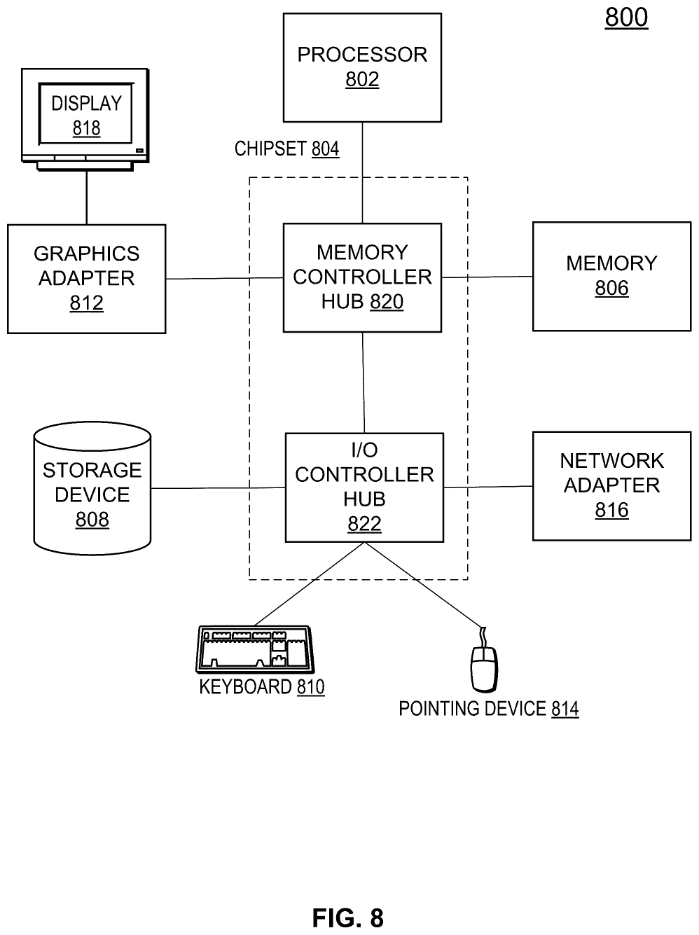

FIG. 8 shows a diagrammatic representation of one embodiment of a computing device in the exemplary form of a computer system 800 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 800 includes a processor 802 and a memory 806 that communicate with each other, and with other components, via a bus. Bus may include any of several types of bus structures including, but not limited to, a memory bus, a memory controller, a peripheral bus, a local bus, and any combinations thereof, using any of a variety of bus architectures.

Processor 802 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 802 may be organized according to Von Neumann and/or Harvard architecture as a non-limiting example. Processor 802 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 806 may include various components (e.g., machine-readable media) including, but not limited to, a random-access memory component, a read only component, and any combinations thereof. In one example, a basic input/output system (BIOS) hub 822, including basic routines that help to transfer information between elements within computer system 800, such as during start-up, may be stored in memory 806. Memory 806 may also include (e.g., stored on one or more machine-readable media) instructions (e.g., software) embodying any one or more of the aspects and/or methodologies of the present disclosure. In another example, memory 806 may further include any number of program modules including, but not limited to, an operating system, one or more application programs, other program modules, program data, and any combinations thereof. Computer system 800 may include memory controller hub 820 which may communicate memory between memory 806, processor 802, and any other components of computer system 800. In some embodiments, computer system 800 includes chipset 804. Chipset 804 may integrate one or more components of computer system 800, such as memory 806 and processor 802.

Computer system 800 may also include a storage device 808. Examples of a storage device (e.g., storage device 808) include, but are not limited to, a hard disk drive, a magnetic disk drive, an optical disc drive in combination with an optical medium, a solid-state memory device, and any combinations thereof. Storage device 824 may be connected to a bus by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 808 (or one or more components thereof) may be removably interfaced with computer system 800 (e.g., via an external port connector (not shown). Particularly, storage device 808 and an associated machine-readable medium may provide nonvolatile and/or volatile storage of machine-readable instructions, data structures, program modules, and/or other data for computer system 800. In one example, software may reside, completely or partially, within a machine-readable medium. In another example, software may reside, completely or partially, within processor 802. Computer system 800 may also include input devices 814, 810. In one example, a user of computer system 800 may enter commands and/or other information into computer system 800 via input devices 814, 810. Examples of input devices 814,810 include, but are not limited to, an alpha-numeric input device (e.g., a keyboard), a pointing device, a joystick, a gamepad, an audio input device (e.g, a microphone, a voice response system, etc.), a cursor control device (e.g., a mouse), a touchpad, an optical scanner, a video capture device (e.g, a still camera, a video camera), a touchscreen, and any combinations thereof. Input devices 814, 810 may be interfaced to a bus via any of a variety of interfaces (not shown) including, but not limited to, a serial interface, a parallel interface, a game port, a USB interface, a FIREWIRE interface, a direct interface to a bus, and any combinations thereof. Input devices 810, 814 may include a touch screen interface that may be a part of or separate from display 818, discussed further below. Input devices 810, 814 may be utilized as a user selection device for selecting one or more graphical representations in a graphical interface as described above.

A user may also input commands and/or other information to computer system 800 via storage device 808 (e.g, a removable disk drive, a flash drive, etc.) and/or network adapter 816. A network interface device, such as network adapter 816, may be utilized for connecting computer system 800 to one or more of a variety of networks. Examples of a network interface device include, but are not limited to, a network interface card (e.g, a mobile network interface card, a LAN card), a modem, and any combination thereof. Examples of a network include, but are not limited to, a wide area network (e.g, the Internet, an enterprise network), a local area network (e.g, a network associated with an office, a building, a campus or other relatively small geographic space), a telephone network, a data network associated with a telephone/voice provider (e.g, a mobile communications provider data and/or voice network), a direct connection between two computing devices, and any combinations thereof. A network may employ a wired and/or a wireless mode of communication. In general, any network topology may be used. Information (e.g, data, software, etc.) may be communicated to and/or from computer system 800 via network adapter 816.

Computer system 800 may further include a graphics adapter 812 for communicating a displayable image to a display device, such as display 818. Examples of a display device include, but are not limited to, a liquid crystal display (LCD), a cathode ray tube (CRT), a plasma display, a light emitting diode (LED) display, and any combinations thereof. Graphics adapter 812 and display 818 may be utilized in combination with processor 802 to provide graphical representations of aspects of the present disclosure. In addition to a display device, computer system 800 may include one or more other peripheral output devices including, but not limited to, an audio speaker, a printer, and any combinations thereof. Such peripheral output devices may be connected to bus a via a peripheral interface. Examples of a peripheral interface include, but are not limited to, a serial port, a USB connection, a FIREWIRE connection, a parallel connection, and any combinations thereof.

### Pill Container Examples

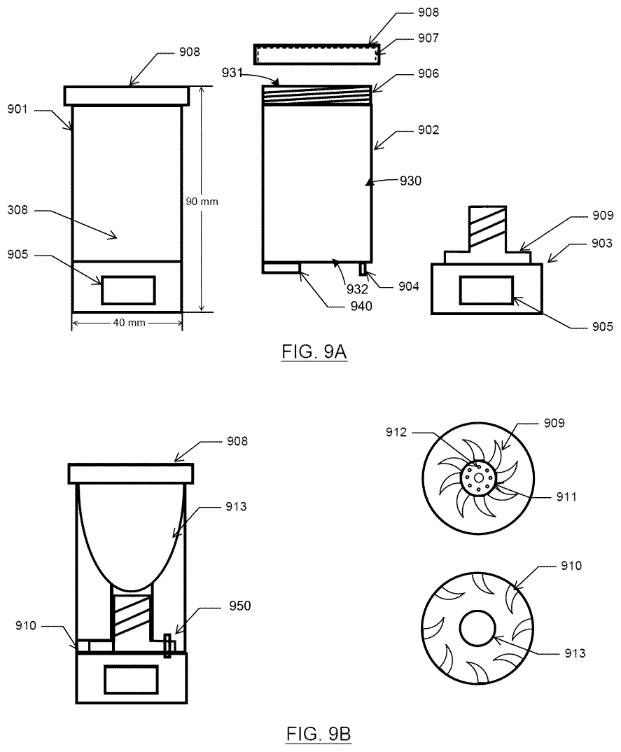

Referring now to FIG. 9A, another embodiment of pill bottle 901 is presented. Again, this may be any type of pill container, but a pill bottle is presented as an example. Pill bottle 901 may be similar to pill bottle 304 as described above with reference to FIG. 3. Pill bottle 901 may be separated into two separate components that fasten together through, for example, a pressure fitting or threading like a screw. The top part 902 may be a hollow structure having one or more side surfaces 930, and the hollow structure may also include a top structure opening 931 and a bottom structure opening 932.

The top part 902 of the pill bottle may contain a hopper that stores the pills before dispensing. The bottom component or enclosure 903 may contain the base, the dispenser, an auger screw and gear teeth. The top part 902 may contain a pawl 940 shown in FIG. 9B, and a shaft 904. The pawl 940 may allow rotation in one direction, but not the other direction similar to a ratchet mechanism. The ratchet mechanism will be unlocked by software and an actuator to move shaft 904. This would allow the ratchet mechanism to dispense one pill per gear tooth 909 interval. The rotation of component 903 will allow one pill from hopper to enter auger screw 911. The pill will move closer to the dispenser 905 with each rotation and be held in the screw chamber by the outer wall of the screw 911. The shaft 904 may interlock with bottom component 903 through periodic holes to disallow rotation and unscheduled dispensing by the user.

An emergency manual override may be implemented to this shaft in scenarios in which a person prescribed a pill needs to take a pill outside of authorized dispensing times. Authorized dispensing times may include times and/or dates of scheduled doses. Unauthorized dispensing times may include times outside of normal dosing scheduling, dispensement outside a locational region, or other unauthorized dispensing times. As a non-limiting example, a user may need to take a breakthrough dose for pain management. Shaft 904 may act as a manual emergency override that causes dispenser 905 to dispense a pill.

An on-board memory of pill bottle 901 and/or software in communication with pill bottle 901 may record activation of shaft 904 and/or dispenser 905, such as times activated, time and date of activation, number of activation attempts, or other information. Dispenser 905 may be similar to or the same as that of dispenser 312 as described above with reference to FIG. 3.

Pill bottle 901 may include threads 906. Threads 906 may be made of plastic or other materials. Threads 906 may have a clockwise or counterclockwise rotation. Threads 906 may be shaped to connect with one or more threads of lid 907. Lid 907 may include a pre-applied thread locker. A pre-applied thread locker may be an adhesive that may secure lid 907 to threads 906. In some embodiments, lid 316 may be secured to threads 320, which may prevent a user from accessing one or more medications in pill bottle 901. Lid 907 may be secured to threads 906 by a medical provider or other medical professional, such as a pharmacist. Lid 907 may also contain an outer lid 908 to assist with tamper proofing. Outer lid 908 may be made of plastic or another similar material to lid 907. Outer lid 908 may rotate freely around lid 907 so that the threads 906 may not be engaged and the bottle may not open.

In some embodiments, pill bottle 901 includes a speaker. A speaker may be piezoelectric, electromagnetic, or other type of speaker. A speaker of pill bottle 901 may be configured to produce a sound. Sounds include beeping, chirping, or any other sound. A speaker of pill bottle 901 may assist in a user finding pill bottle 901 when pill bottle 901 is lost. For instance, through software in communication with pill bottle 901, a user may request that pill bottle 901 plays a sound through a speaker to aid in finding pill bottle 901. In some embodiments, pill bottle 901 has a LED configured to flash a bright light that may assist in a user finding pill bottle 901 when pill bottle 901 is lost. A user may request through software in communication with pill bottle 901 aid in finding pill bottle 901 when pill bottle 901 is lost. Software may cause a speaker and/or LED light of pill bottle 901 to activate, which may cause pill bottle 901 to play a sound, emit a bright light, or combinations thereof. Software in communication with pill bottle 901 may display an indicator of pill bottle 901 through a display device of a computing device. For instance, an arrow, radius, or other graphical indicator may be displayed through a display device of a computing device in communication with software. In some embodiments, software may generate an augmented reality (AR) display that indicates where pill bottle 901 is located when lost. For instance, software may display real-time video of the surroundings of a computing device in communication with the software with a graphical icon superimposed in a three-dimensional mapping of a user's surroundings. Graphical images may include, but are not limited to, pills, bottles, or other symbols. A user may continually activate one or more speakers, lights, or other components of pill bottle 901 through a computing device in communication with software and pill bottle 901. In some embodiments, pill bottle 901 has a location device such as a GPS tag, ultra-band tag, or other device. Pill bottle 901 may communicate locational data of itself through a communication module based on locational data generated by a locational device. Software in communication with pill bottle 901 may determine a last known location of pill bottle 901 and may communicate the last known location of pill bottle 901 to a user through a computing device in communication with the software, which may aid a user in finding pill bottle 901 when pill bottle 901 is lost.

Referring now to FIG. 9B, an additional embodiment of pill bottle 901 is presented. The bottle consists of an internal funnel 913, a rotating screw on lid 908, a rotating disc at the bottom of the bottle, and a locking pin 950. To the right there are two possible variants of rotating disc. As main disc 912 rotates at the bottom of the bottle, grooves 910 channel pills from the inward out that land from above funnel 913. In the figure below, as rotating disc rotates, grooves 910 facing the opposite direction may funnel pills from outward in.

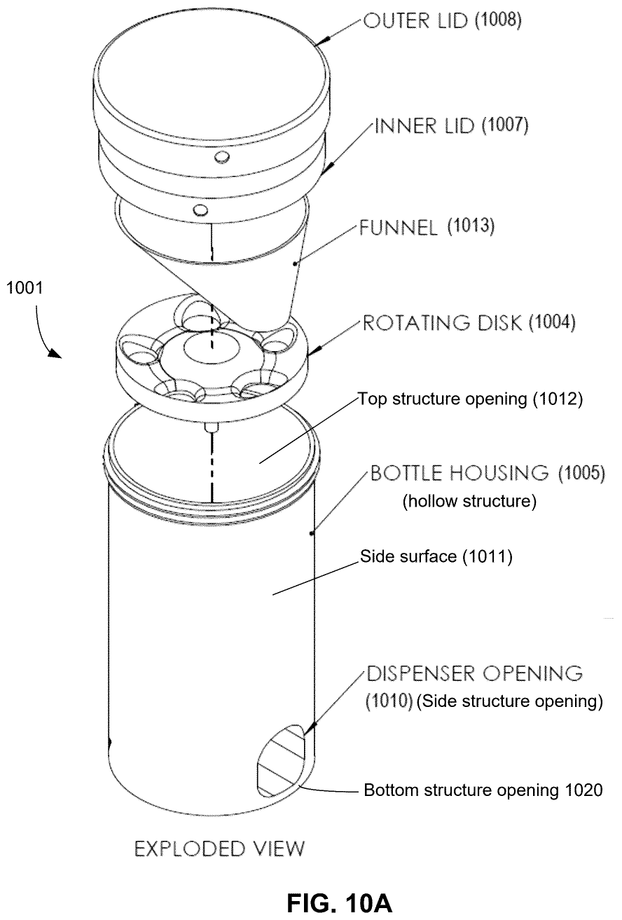

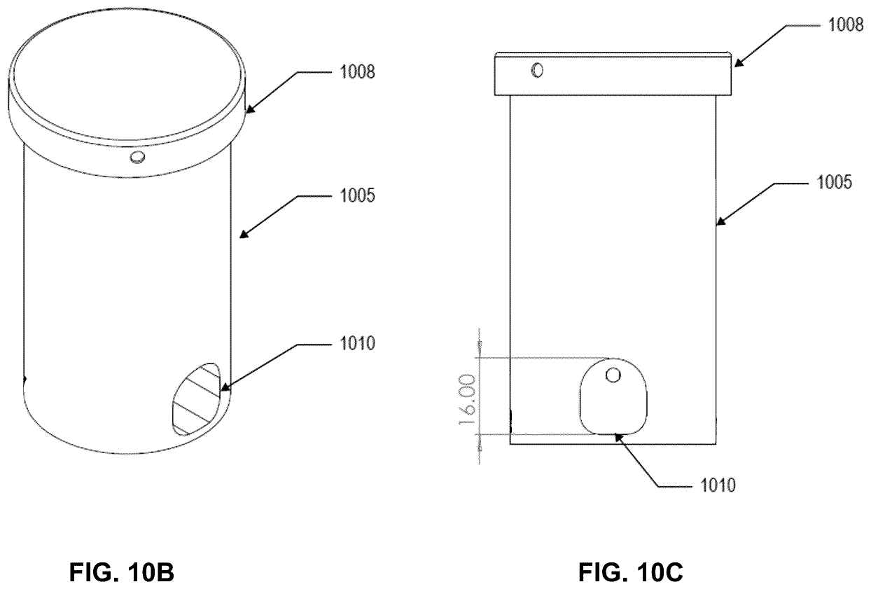

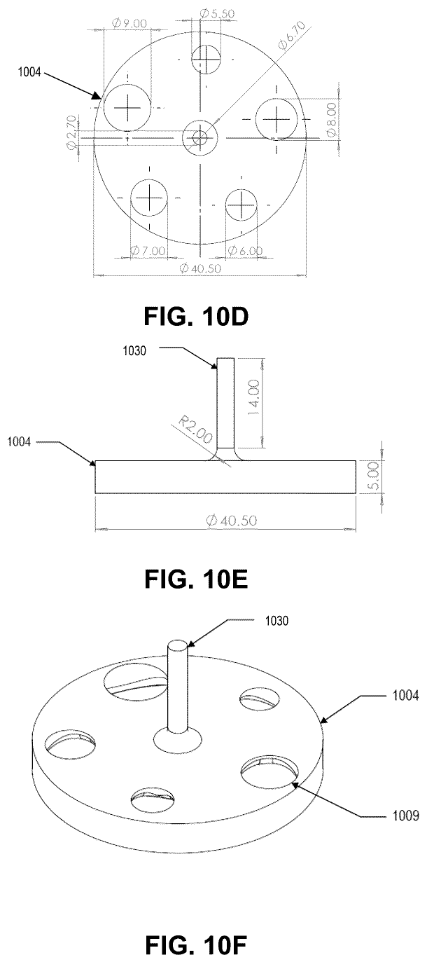

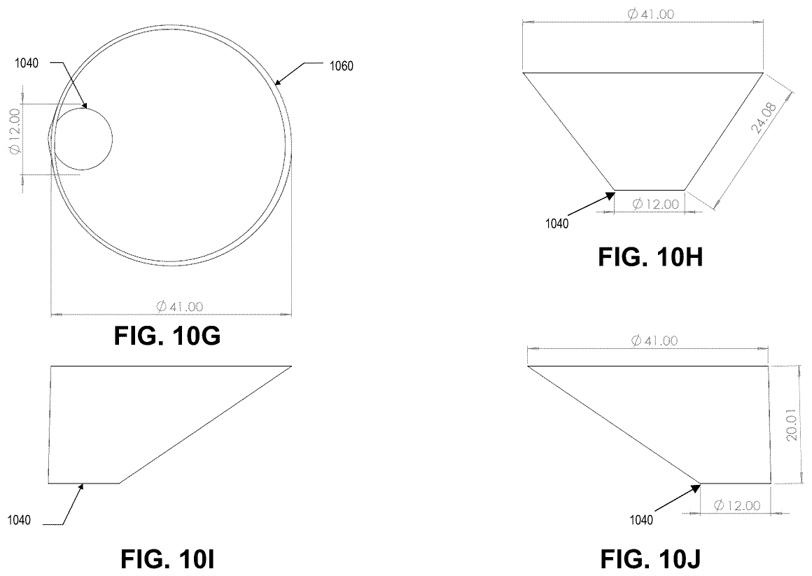

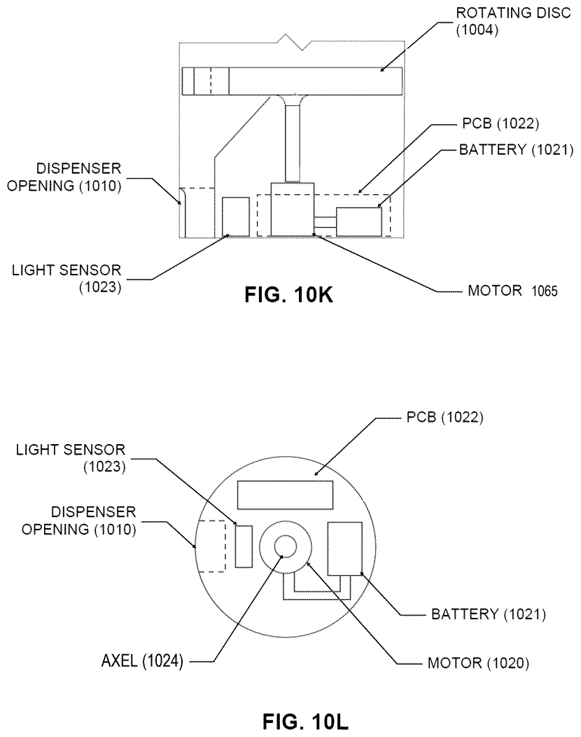

FIG. 10A provides an exploded view of another embodiment of the pill bottle or pill container 1001 (which, with other features, such as the image detection, may be referred to as the pill container system or pill bottle container). It may include any of the features described with regard to the pill bottles above. This may be a top part of the pill container similar to the top part 902 shown in FIG. 9A. This top part may fit on a base or enclosure like the base/enclosure 903 shown in FIG. 9A, but in the case of FIG. 10A, the enclosure (not shown) may contain electronics associated with the pill container rather than having the dispenser opening as is shown in FIG. 9A. The dispenser opening or side structure opening 1010 in FIG. 10A is in the top part of the pill container 1001, above the enclosure on top of which the top part of the pill container sits. Pill container 1001 may be separated into several separate components that are set in place through pressure fitting or screw threading, similar to that shown in FIG. 9A.

As FIG. 10A shows, the overall pill container 1001 assembly features bottle housing 1005 (or hollow structure), side surface 1011, top structure opening 1012, bottom structure opening 1020, dispenser opening (or side structure opening) 1010, rotating disc 1004, funnel 1013, inner lid 1007, and outer lid 1008.

The bottle housing 1005 may be referred to as a hollow structure or hollow tube. This housing 1005 may envelope the parts of the pill container system and help keep the pills secure and the components together. The hollow structure or housing 1005 includes a side surface 1011 or side wall (or can include more than one side surface). The hollow structure has a top structure opening 1012 at its top in FIG. 10A, a bottom structure opening 1020 at its bottom in FIG. 10A, and a dispenser opening or side structure opening 1010 at a side of the hollow structure. The dispenser/side structure opening 1010 extends through one of the one or more side surfaces 1011. The top structure opening 1012 is open to allow pills to be inserted into the container. The bottom structure opening 1020 is open to allow it to attach to an enclosure (not shown) that may house electronics or other components of the pill bottle. In other designs, the electronics are housed elsewhere, such as in the lid. The bottom may also be closed in some embodiments, or may be partially closed, or may be closed but include one or more openings in a bottom surface.

The dispenser/side structure opening 1010 may be opened and closed or covered by a door that opens and closes it. The electronics system may control the opening and closing of the dispenser to dispense pills to the user at the appropriate time, as described above in detail for the other example pill bottle embodiments.

The pill container system includes a locking lid system, which is comprised of the outer lid 1008 and the inner lid 1007. The locking lid is attachable to hollow structure such that the top structure opening 1012 is covered when the locking lid is attached. The lid design may be similar to the lid designs presented above for other example pill bottle embodiments. The lid is described in more detail below.

In some embodiments, the pill container system includes an enclosure (e.g., bottom portion 903) configured to attach at the bottom structure opening 1020. The enclosure is detachable from the hollow structure and houses an electronic system (including as the controller described herein) configured to control opening and closing of the door for dispensing pills from the side structure opening 1010. The top part or housing can be detached and replaced with another housing that is attached to the enclosure, or the housing can be detached, cleaned, and replaced onto the enclosure. The housing may be disposable.

In some embodiments, the electronic system includes a processor and a non-transitory computer-readable storage medium. The electronic system may receive an indication that a client device verified the identity of a user associated with the pill container system using one or more verification mechanisms, such as analysis of image data, receipt of a passcode, etc. Examples of the systems and methods that can be used were presented above, including in FIGS. 4, 6, and 7B. The electronic system may cause the door to open such that a pill is dispensed from the pill container system upon determining that the user is associated with a prescription of the pill container system.

FIG. 10B illustrates an isometric view of the pill container 1001 in accordance with one or more embodiments. This view shows the outer lid 1008 on top of the housing or hollow structure 1005 and the dispenser or side structure opening 1010 from which pills may be dispenses.

FIG. 10C illustrates a side view of the pill container 1001, in accordance with one or more embodiments. This view illustrates the same components as FIG. 10B, but it also provides an example of a measurement of the dispenser opening 1010. In this example, the opening is 16 mm in height, though it can be larger or smaller.

FIG. 10D shows a representation of rotating surface or rotating disk 1004 from a top view. This rotating surface is shown as a disk in this example, but it could be other shapes. The rotating disk 1004 is positioned within the hollow structure or housing (above the side structure opening) and it can rotate about an axis that extends through the top opening and bottom opening and extends perpendicular to the side walls or surfaces of the housing/hollow structure. and. The rotating disk may have one or more slots 1009 or openings of varying sizes. The use of the different sizes of openings or slots 1009 allows controlled dispensing of pills of varying sizes. The rotating surface or disk has stem or shaft 1030 that passes through the bottle housing into the lower cavity, so that it may engage with an electric motor or other turning device.

FIG. 10E illustrates a side view of the rotating disk 1004. The side view shows the shaft 1030 extending upward from the center of the top surface of the rotating disk 1004.

FIG. 10F illustrates an isometric view of the rotating disk 1004. The shaft 1030 or axle is centered between the openings or slots 1009 in the rotating disk 1004. The slots 1009 vary in size from a largest to a smallest size. There can be one or more slots designed to allow pills of different sizes to pass through. The openings are shown as circular, but they can be different shapes and can differ from one another in shape.

FIG. 10G shows a representation of the hopper or the funnel 1013 from a top view. The funnel 1013 may have a larger diameter circular opening at the top 1060, and a smaller diameter circular opening at bottom 1012 (smaller relative to the larger diameter opening). The openings are circular in this example, but could be other shapes. The funnel could also be shaped differently. The openings may or may not be concentric such that the funnel 1013 moves the pills together and to one side onto rotating disk 1004. For example, FIG. 10H shows the funnel 1013 from a first side view, where the centers of the openings appear aligned. However, from second side view of FIG. 101 and third side view of FIG. 10J, the center of the smaller diameter circular opening at bottom 1012 is offset from the center of the larger diameter circular opening at the top 1060.

The hopper or funnel 1013 stores the pills before dispensing. This funnel may be fixed in place using various methods, including but not limited to pressure fitting adhesive, etc. The end of the funnel tapers down to a funnel opening 1040 to dispense a single pill at a time. During dispensing, this opening 1012 aligns with the appropriate slot 1009 of the rotating disk 1004 corresponding to the input pill size. This is done through the rotation of the rotating disk 1004 around a shaft 1030 by a motor or actuator.

A sensor may be included after the rotating disk 1004 to ensure a pill has been dispensed out of the dispenser 1010. This sensor may be positioned on the rotating disk 1004, or on any surface inside the housing of the pill container. Once the desired amount of pills is dispensed, the motor will return the rotating disk 1004 back to a closed position. This closed position aligns the funnel opening 1040 so that it is positioned in between openings 1009 of the rotating disk 1004 and so it cannot allow pills to be dispensed. The pill dispensing is blocked by the rotating disk 1004 surface.

An emergency manual override may be implemented to the shaft 1030 of the rotating disk 1004 in scenarios in which a person prescribed a pill needs to take a pill outside of authorized dispensing times. Authorized dispensing times may include times and/or dates of scheduled doses. Unauthorized dispensing times may include times outside of normal dosing scheduling, dispensement outside a locational region, or other unauthorized dispensing times. As a non-limiting example, a user may need to take a breakthrough dose for pain management. An on-board memory of pill container 1001 and/or software in communication with pill container 1001 may record the rotation of the rotating disk 1004, such as times activated, time and date of activation, number of activation attempts, or other information.

Pill container 1001 may include threads 1006. Threads 1006 may be made of plastic or other materials. Threads 1006 may have a clockwise or counterclockwise rotation. Threads 1006 may be shaped to connect with one or more threads of lid 1007. Lid 1007 may include a pre-applied thread locker. A pre-applied thread locker may be an adhesive that may secure lid 1007 to threads 1006. In some embodiments, lid 1007 may be secured to threads 1006, which may prevent a user from accessing one or more medications in pill container 1001. Lid 1007 may be secured to threads 1006 by a medical provider or other medical professional, such as a pharmacist. Lid 1007 may also contain an outer lid 1008 to assist with tamper proofing. Outer lid 1008 may be made of plastic or another similar material to lid 1007. Outer lid 1008 may rotate freely around lid 1007 so that the threads 1006 may not be engaged and the bottle may not open.

In some embodiments, pill container 1001 includes a speaker. A speaker may be piezoelectric, electromagnetic, or other type of speaker. A speaker of pill container 1001 may be configured to produce a sound. Sounds include beeping, chirping, or any other sound. A speaker of pill container 1001 may assist in a user finding pill container 1001 when pill container 1001 is lost. For instance, through software in communication with pill container 1001, a user may request that pill container 1001 plays a sound through a speaker to aid in fining pill container 1001. In some embodiments, pill container 1001 has an LED configured to flash a bright light that may assist in a user finding pill container 1001 when pill container 1001 is lost. A user may request through software in communication with pill container 1001 aid in finding pill container 1001 when pill container 1001 is lost. Software may cause a speaker and/or LED light of pill container 1001 to activate, which may cause pill container 1001 to play a sound, emit a bright light, or combinations thereof. Software in communication with pill container 1001 may display an indicator of pill container 1001 through a display device of a computing device. For instance, an arrow, radius, or other graphical indicator may be displayed through a display device of a computing device in communication with software. In some embodiments, software may generate an augmented reality (AR) display that indicates where pill container 1001 is located when lost. For instance, software may display real-time video of surroundings of a computing device in communication with the software with a graphical icon superimposed in a three-dimensional mapping of a user's surroundings. Graphical images may include, but are not limited to, pills, bottles, or other symbols. A user may continually activate one or more speakers, lights, or other components of pill container 1001 through a computing device in communication with software and pill container 1001. In some embodiments, pill container 1001 has a location device such as a GPS tag, ultra-band tag, or other device. Pill container 1001 may communicate locational data of itself through a communication module based on locational data generated by a locational device. Software in communication with pill container 1001 may determine a last known location of pill container 1001 and may communicate the last known location of pill bottle 901 to a user through a computing device in communication with the software, which may aid a user in finding pill container 1001 when pill container 1001 is lost.

FIG. 10K illustrates a side view of a bottom portion of a pill bottle container, according to one or more embodiments. The bottom portion may include the rotating disc 1004, printed circuit board (PCB) 1022, a motor 1065, a dispenser opening 1010, and a light sensor 1023. The motor, when powered, may cause the rotating disc 1004 to rotate such that a pill may move through a slot of the rotating disc 1004. The light sensor 1023 may capture data describing a change in light, which may be indicative of a pill exiting through the dispenser opening. In some embodiments, the bottom portion is the same as the bottom portion 903 described in relation to FIG. 9 and is able to attach and unattach from the rest of the pill bottle container such that different bottoms portions can attach to different hollow structures. The rotating disc 1004 may be included in the bottom portion, as is shown in FIG. 10K. In some embodiments, the rotating disc remains in the hollow structure of the rest of the pill bottle container when the bottom portion is removed from the rest of the pill bottle container.

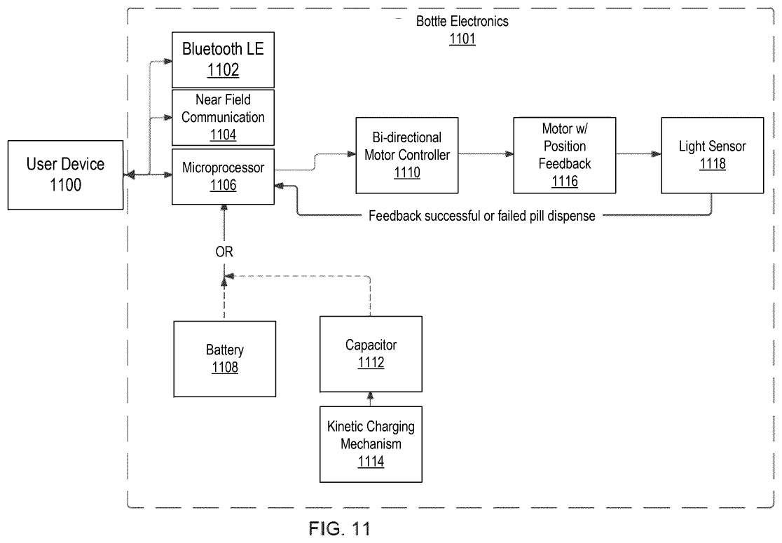

FIG. 10L illustrates a top view of a bottom portion of a pill bottle container, according to one or more embodiments. As described in relation to FIG. 10K, the bottom portion may include a dispenser opening 1010, motor 1065, battery 1021, PCB 1022, light sensor 1023, and axel 1024. The axel 1024 may be part od the rotating disc 1004 and, in some embodiments, may extend into the hollow structure of the rest of the pill bottle container. The light sensor 1023 may be positioned at or near the dispenser opening 1010 such that the light sensor 1023 can capture data indicative of light through the dispenser opening 1010. Data indicative of a decrease in light may represent a pill exiting the pill bottle container through the dispenser opening 1010. The PCB 1022, battery 1021, and motor 1065 may be part of bottle electronics of the pill bottle container. Pill bottle electronics are further described in relation to FIG. 11.

FIG. 11 is a block diagram of bottle electronics 1101 for a pill bottle container, according to one or more embodiments. The bottle electronics 1101 may be housed in the bottom portion of the pill bottle container. The bottle electronics 1101 includes Bluetooth LE 1102, near field communication 1104, microprocessor 1106, battery 1108, bi-directional motor controller 1110, capacitor 1112, kinetic charging mechanism 1114, motor with position feedback 1116, and light sensor 1118. A user device may be in communication with the bottle electronics 1101. The user device may be of varying types (ex. iOS device, Android Device, or other internet connected device). A user device 1100 may communicate with the bottle electronics 1101 using Bluetooth LE 1102 or near field communication 1104. Central logic within the bottle electronics 1101 may consist of a microprocessor 1106. The power source of the pill bottle container may be a rechargeable lithium-ion battery 1108 or another type of battery 1108. The power source of the bottle electronics 1101 also may be a capacitor 1112 for short-term energy storage. This capacitor 1112 may require application of kinetic energy by the user for the kinetic charging mechanism 1114. For example, shaking of the pill bottle container to provide an amount of energy for the pill bottle container to complete its operation. Once data has been exchanged between the user device 1100 and microprocessor 1106 of the bottle electronics 1101, the microprocessor 1106 may then command a motor controller to provide energy to an electric motor 1116 to turn in one direction or the other. This motor 1116 may have position sensing features to report the rotational position back to the microprocessor 1106. A light sensor 1118 may also be present to detect light as a dispensed pill exits the pill bottle container through the dispenser opening. This light sensor 1118 may then feed information back to the microprocessor 1106 to relay successful or failed dispensing of pill through actuation of the electric motor 1116. The microprocessor 1106 then may relay this information back to the user device 1100, which may relay this information.

Representative embodiments are described above. It will be understood that reasonable equivalents to the embodiments described above, or to the elements of the embodiments described above, are consistent with practicing the present invention and included in the present disclosure.

### Additional Considerations

As used herein, the term “includes” followed by one or more elements does not exclude the presence of one or more additional elements. The term “or” should be construed as a non-exclusive “or” (e.g., “A or B” may refer to “A,” “B,” or “A and B”) rather than an exclusive “or.” The articles “a” or “an” refer to one or more instances of the following element unless a single instance is clearly specified.

The drawings and written description describe example embodiments of the present disclosure and should not be construed as enumerating essential features of the present disclosure. The scope of the invention should be construed from any claims issuing in a patent containing this description.

Further, some figures depict one or more measurements of components in the figures. While such values represent example measurements of the components, these values are examples and other sizes are also possible.

1. A pill container comprising:
a hollow structure comprising:
one or more side surfaces,
a top structure opening,
a bottom structure opening, and
a side structure opening that extends through one of the one or more side surfaces;
a rotating surface configured to be positioned within the hollow structure and above the side structure opening and to rotate about a first axis that extends perpendicular to the one or more side surfaces and through the top opening and bottom opening, wherein the rotating surface includes one or more slots that extend through the rotating surface; and
an enclosure configured to attach at the bottom structure opening of the hollow structure, wherein the enclosure is detachable from the hollow structure and houses an electronic system configured to control rotation of the rotating surface.

2. The pill container of claim 1, wherein the one or more slots are positioned along a ring around the first axis, the pill container further comprising:
a funnel positioned within the hollow structure above the rotating surface, wherein a top funnel opening of the funnel spans a width between the one or more surfaces and a bottom funnel opening of the funnel is aligned above the ring of one or more slots.

3. The pill container of claim 2, wherein the enclosure includes a sensor embedded in a top portion of the enclosure, wherein the sensor is aligned with the bottom funnel opening of the funnel when the enclosure is attached to the hollow structure, the sensor configured to capture signals indicative of a pill moving through the side structure opening of the hollow structure.

4. The pill container of claim 3, wherein the sensor is a light sensor, pressure sensor, or a motion sensor.

5. The pill container of claim 1, further comprising:
a lid configured to attach at the top structure opening via a locking mechanism, wherein the lid is rotatable around the first axis while attached at the top structure opening.

6. The pill container of claim 1, wherein the one or more slots include at least two slots, each slot a different size from the other slots of the one or more slots.

7. The pill container of claim 6, wherein a first slot of the one or more slots is configured to allow pills of a first size and a second size to move through the first slot and a second slot of the one or more slots is smaller and is configured to allow pills of the first size to move through the second slot and to prevent pills of the second size from moving through the second slot.

8. The pill container of claim 1, wherein the electronic system is configured to receive a signal from a client device and cause the rotating surface to rotate such that one or more pills are dispensed through the side structure opening in response to receiving the signal.

9. A method comprising:
sending, to a pill container from a client device, a signal to dispense a first pill;
causing a camera of the client device to capture image data;
inputting the image data to a machine-learned model, wherein the machine-learned model is trained to determine whether the image data depicts a user associated with the pill container swallowing the first pill;
receiving, from the machine-learned model, an indication of whether the image data depicts the user swallowing the first pill; and
in response to determining that the image data depicts the user swallowing the first pill, storing a first set of compliance data in association with an identifier of the user, wherein the first set of compliance data indicates that the user swallowed the first pill.

10. The method of claim 9, further comprising:
determining that a current time at the client device is in a time window for dispensing a second pill; and
in response to determining that a most recently stored set of compliance data is indicative of the user swallowing a pill, sending a second signal to dispense a second pill.

11. The method of claim 9, further comprising:
in response to determining that the image data does not depict the user swallowing the first pill, sending an alert to a second client device associated with a medical entity; and
storing a second set of compliance data in association with the identifier of the user, wherein the second set of compliance data indicates that the user did not swallow the first pill.

12. The method of claim 11, further comprising:
determining that a current time at the client device is in a time window for dispensing a second pill; and
in response to determining that a most recently stored set of compliance data indicates that the user did not swallow a pill, sending an alert to a medical entity associated with the user.

13. The method of claim 9, wherein the indication of whether the image data depicts the user swallowing the pill comprises indication of a likelihood that the user swallowed the first pill, the method further comprising:
triggering an alert in response to the likelihood being outside a threshold tolerance.

14. The method of claim 13, wherein a first class of pills are associated with a lower threshold tolerance than a second class of pills.

15. The method of claim 9, further comprising:
training the machine-learned model on a first set of labeled image data, wherein image data depicting one or more of a set of throat movements is labeled as depicting swallowing, and image data not depicting one or more of the set of throat movements is labeled as not depicting swallowing; and
training the machine-learned model on a second set of labeled image data, wherein image data of the user is labeled as depicting the user and image data that is not of the user is labeled as not depicting the user.

16. The method of claim 9, further comprising:
receiving, via a user interface at the client device, an indication that the user lost the pill container; and
sending, to a medical entity associated with the user, an indication that the user lost the pill container.

17. The method of claim 9, further comprising:
receiving, via a user interface at the client device, an indication that the user lost the pill container;
requesting, from the pill container, a current location of the pill container;
comparing the current location of the pill container to a current location of the client device; and
in response to the current location of the pill container being outside of a threshold distance from the current location of the client device, sending a request for new pills to a second client device associated with a medical entity.

18. A method comprising:
receiving, at a pill container from a client device, an indication to dispense a pill from the pill container, wherein the pill container contains at least one pill in a first enclosure, the first pill positioned above a rotating surface within the pill container;
causing the rotating surface within the pill container to rotate, wherein the rotating surface includes a plurality of slots, wherein at least one slot is wide enough for the pill to move through the slot; and
in response to detecting, via a sensor configured at an opening of the pill container, a signal indicative of a pill moving through the opening, wherein the opening is below the rotating surface:
sending, to the client device, an indication that the pill was dispensed; and
stopping rotation of the rotating surface.

19. The method of claim 18, wherein each slot of the plurality of slots has a different width than the other slots.

20. The method of claim 18, further comprising:
sending, for integration in an electronic record of an electronic record system associate with a medical entity, a notification that the pill was dispensed at the pill container.
